(19) United States
(12) Patent Application Publication
(10) Pub. No.: US 2026/0224647 A1
(43) Pub. Date: Aug. 6, 2026

(54) Microbial compositions for treating joint inflammation

(71) Applicants: Katholieke Universiteit Leuven, Leuven (BE); MRM Health N.V., Gent (BE); Universiteit Gent, Gent (BE); VIB vzw, Gent (BE)

(72) Inventors: Iris Pinheiro, Gent (BE); Jolien Benedict Souffriau, Harelbeke (BZ); Nicole Reichardt, Zwijnaarde (BE); Sam Possemiers, Zwijnaarde (BE); Selin Bolca, Zwijnaarde (BE); Suchita Panda, Merelbeke (BE); Anaïs Biclot, Leuven (BE); Dirk Elewaut, Gent (BE); Emilie Dumas, Gent (BE); Geert R.B. Huys, Leuven (BE); Jeroen Raes, Leuven (BE); Jorge Francisco VAZQUEZ CASTELLANOS, Leuven (BE); Lucas Ferreira Maciel, Leuven (BE)

(73) Assignees: Katholieke Universiteit Leuven, Leuven (BE); MRM Health N.V., Gent (BE); Universiteit Gent, Gent (BE); VIB vzw, Gent (BE)

(21) Appl. No.: 19/150,343

(22) PCT Filed: Jan. 29, 2024
(86) PCT No.: PCT/EP2024/052103

§ 371 Date: Jul. 23, 2025

(30) Foreign Application Priority Data
Jan. 30, 2023 (EP) 23153960.2

(51) Int. Cl. A61K 35/747; A61P 19/02; A61P 29/00

(52) U.S. Cl. CPC A61K 35/747; A61P 19/02; A61P 29/00

(57) ABSTRACT

Described herein is a composition of two or more bacterial strains, wherein the composition comprises a bacterial strain of at least two of the following genera: Phascolarctobacterium, Veillonella, Eubacterium, Agathobacter, Lactiplantibacillus, and Anaerostipes. Aspects and embodiments described herein may be used in the treatment of joint inflammation, symptoms thereof, and underlying diseases and conditions.

### FIELD

Aspects and embodiments described herein relate to the field of microbial compositions for human health, in particular joint inflammation and associated diseases and conditions.

### BACKGROUND

Several chronic diseases and conditions including rheumatoid arthritis (RA), juvenile idiopathic arthritis (JIA), spondyloarthritis (SpA), lupus, and Lyme disease, are characterized by joint inflammation as a common element. If joint inflammation is left untreated, it invariably leads to joint damage and deformities, and patients may become disabled as a result. Although several treatment options are available, none of these are as safe and effective as could be desired.

The role of the intestinal microbiota in joint inflammation and associated diseases and conditions is a growing field of study.

In a proof-of-concept clinical study (NCT03058900), the safety and efficacy of fecal microbiota transplantation (FMT) in psoriatic arthritis (PsA), a type of spondyloarthritis, has been evaluated. It was concluded that FMT appeared to be inferior to sham in treating active peripheral PsA (see Kragsnaes et al. Safety and efficacy of faecal microbiota transplantation for active peripheral psoriatic arthritis: an exploratory randomised placebo-controlled trial. Ann Rheum Dis 2021; 80(9):1158-1167).

Additionally, the fact that fecal transfer or fecal microbial transplantation (FMT) is a poorly characterized procedure comes with transmission risks of infectious diseases and raises doubts about its applicability in less acute and life-threatening pathologies. Indeed, FMT is unlikely to become a long-term therapeutic option, especially considering the chronic and progressive nature of most instances of joint inflammation.

Initial attempts based on defined, orally-administered probiotics were unsuccessful. Hattaka et al. reported that patients receiving twice daily administration of Lactobacillus rhamnosus GG (LGG) showed no statistically significant differences in the activity of rheumatoid arthritis (see Hatakka et al. Effects of probiotic therapy on the activity and activation of mild rheumatoid arthritis—a pilot study. Scand J Rheumatol 2003; 32(4):211-215). Brophy et al. reported that daily administration of a probiotic consisting of Lactobacillus salivarius, Lactobacillus paracasei, Bifidobacterium infantis, and Bifidobacterium bifidum showed no statistical or clinical significant improvement in SpA (Brophy et al. Internet-based randomised controlled trials for the evaluation of complementary and alternative medicines: probiotics in spondyloarthropathy. BMC Musculoskelet Disord 2008; 9:4). Jenks et al. reported that daily administration of a probiotic consisting of Streptococcus salivarius, Bifidobacterium lactis, and Lactobacillus acidophilus did not demonstrate significant benefit over placebo in patients with active spondyloarthritis (Jenks et al. Probiotic Therapy for the Treatment of Spondyloarthritis: A Randomized Controlled Trial. J Rheumatol 2010; 37(10):2118-2125). Overall, a recent meta-analysis concluded that (combined) formulations of Bifidobacteriales and Lactobacillales may provide small but statistically significant benefits in RA and SpA (see Lowe et al. A systematic review of the effects of probiotic administration in inflammatory arthritis. Complement Ther Clin Pract 2020; 40:101207).

### SUMMARY

In view of the above, there is still a need for improved means and methods which can be used for treating and preventing joint inflammation and associated conditions and symptoms, and which do not have all the drawbacks of existing therapies.

As described in this disclosure, it has been surprisingly found that specific combinations of bacteria have particular and surprising beneficial effects. Particularly, as elaborated elsewhere herein and in the experimental part, it has been found that bacterial compositions as described herein exert a therapeutic effect on joint inflammation.

Accordingly, the aspects and embodiments described herein solve at least some of the problems and needs as discussed herein.

An aspect of this disclosure relates to a composition of two or more bacterial strains, wherein the composition comprises a bacterial strain of at least two of the following genera: Phascolarctobacterium, Veillonella, Eubacterium, Agathobacter, Lactiplantibacillus, and Anaerostipes, for use in a method of treating or preventing joint inflammation, a symptom thereof, or an underlying disease or condition. In some embodiments, a composition for use according to the disclosure is such that the composition comprises a bacterial strain of at least two of the following species: Phascolarctobacterium faecium, Veillonella atypica, Agathobacter rectalis, Lactiplantibacillus plantarum, and Anaerostipes caccae. In some embodiments, a composition for use according to the disclosure is such that the composition comprises a bacterial strain of the genus Veillonella and a bacterial strain of the genus Anaerostipes, preferably wherein the composition comprises a bacterial strain of the species Veillonella atypica and a bacterial strain of the species Anaerostipes caccae.

In some embodiments, a composition for use according to the disclosure is such that the composition comprises a bacterial strain of at least three or at least four of the following genera: Phascolarctobacterium, Veillonella, Eubacterium, Agathobacter, Lactiplantibacillus, and Anaerostipes. In some embodiments, a composition for use according to the disclosure is such that the composition comprises a bacterial strain of at least three or at least four of the following species: Phascolarctobacterium faecium, Veillonella atypica, Agathobacter rectalis, Lactiplantibacillus plantarum, and Anaerostipes caccae. In some embodiments, a composition for use according to the disclosure is such that the composition comprises a bacterial strain of the genus Veillonella, a bacterial strain of the genus Anaerostipes, and a bacterial strain of the genus Phascolarctobacterium, preferably wherein the composition comprises a bacterial strain of the species Veillonella atypica, a bacterial strain of the species Anaerostipes caccae, and a bacterial strain of the species Phascolarctobacterium faecium. In some embodiments, such composition for use comprising a bacterial strain of the genus Veillonella, a bacterial strain of the genus Anaerostipes, and a bacterial strain of the genus Phascolarctobacterium is such that the composition further comprises a bacterial strain of the genus Agathobacter, preferably of the species Agathobacter rectalis. In some embodiments, such composition for use comprising a bacterial strain of the genus Veillonella, a bacterial strain of the genus Anaerostipes, and a bacterial strain of the genus Phascolarctobacterium is such that the composition further comprises a bacterial strain of the genus Lactiplantibacillus, preferably of the species Lactiplantibacillus plantarum.

In some embodiments, a composition for use according to the disclosure is such that the composition comprises a bacterial strain of at least five of the following genera: Phascolarctobacterium, Veillonella, Eubacterium, Agathobacter, Lactiplantibacillus, and Anaerostipes, preferably wherein the composition comprises a bacterial strain of each of the following genera: Phascolarctobacterium, Veillonella, Agathobacter, Lactiplantibacillus, and Anaerostipes. In some embodiments, a composition for use according to the disclosure is such that the composition comprises a bacterial strain of each of the following species: Phascolarctobacterium faecium, Veillonella atypica, Agathobacter rectalis, Lactiplantibacillus plantarum, and Anaerostipes caccae. In some embodiments, a composition for use according to the disclosure is such that the composition comprises Phascolarctobacterium faecium LMG P-32922, Veillonella atypica LMG P-32913, Agathobacter rectalis DSM 34506, Lactiplantibacillus plantarum LMG P-29366, and Anaerostipes caccae LMG P-29359.

In some embodiments, a composition for use according to the disclosure is such that the underlying disease or condition is inflammatory arthritis, preferably autoimmune inflammatory arthritis. In some embodiments, the autoimmune inflammatory arthritis is selected from the group consisting of: rheumatoid arthritis (RA), juvenile idiopathic arthritis (JIA), spondyloarthritis (SpA), lupus, Sjögren syndrome, and Lyme disease. In some embodiments, the autoimmune inflammatory arthritis is spondyloarthritis (SpA).

In some embodiments, a composition for use according to the disclosure is such that the method of treating or preventing spondyloarthritis further comprises the use of a therapeutic agent selected from the group consisting of non-steroidal anti-inflammatory drugs (NSAIDs), glucocorticoids and disease-modifying antirheumatic drugs (DMARDs). In some embodiments, the method of treating or preventing spondyloarthritis further comprises the use of a DMARD. In some embodiments, the DMARD is a TNF inhibitor.

A further aspect of this disclosure relates to a composition comprising a bacterial strain of each of the following genera: Phascolarctobacterium, Veillonella, Agathobacter, and Anaerostipes, preferably comprising a bacterial strain of each of the following species: Phascolarctobacterium faecium, Veillonella atypica, Agathobacter rectalis, and Anaerostipes caccae. In some embodiments, a composition according to the disclosure is such that it comprises Phascolarctobacterium faecium LMG P-32922, Veillonella atypica LMG P-32913, Agathobacter rectalis DSM 34506, and Anaerostipes caccae LMG P-29359.

A further aspect of this disclosure relates to a composition comprising a bacterial strain of each of the following genera: Phascolarctobacterium, Veillonella, Agathobacter, Lactiplantibacillus, and Anaerostipes, preferably comprising a bacterial strain of each of the following species: Phascolarctobacterium faecium, Veillonella atypica, Agathobacter rectalis, Lactiplantibacillus plantarum, and Anaerostipes caccae. In some embodiments, a composition according to the disclosure is such that it comprises Phascolarctobacterium faecium LMG P-32922, Veillonella atypica LMG P-32913, Agathobacter rectalis DSM 34506, Lactiplantibacillus plantarum LMG P-29366, and Anaerostipes caccae LMG P-29359.

### Deposit of Biological Material

Purified cultures of the microbial strains Lactiplantibacillus plantarum LMG P-29366 (also denoted as Lactiplantibacillus plantarum PD15) and Anaerostipes caccae LMG P-29359 (also denoted as Anaerostipes caccae PD10) described in the present application were deposited by depositor Prodigest (Technologiepark 3, 9052 Zwijnaarde, Belgium; current address: Technologiepark-Zwijnaarde 82, 9052 Gent, Belgium) with the Belgian Coordinated Collections of Microorganisms (BCCM) (BCCM/LMG Bacteria collection, Universiteit Gent, Laboratorium voor Microbiologie, K. L. Ledeganckstraat 35, 9000 Gent, Belgium), recognized as an International Depositary Authority by the Budapest Treaty and the World Intellectual Property organization. Original deposits have been made on 18 Jan. 2016.

Purified cultures of the microbial strain Agathobacter rectalis DSM 34506 (also denoted as Agathobacter rectalis DGCC14098) described in the present application were deposited by depositor DuPont Nutrition Biosciences ApS (Parallelvej 16, 2800 Kongens Lyngby, Denmark) with Leibniz-Institut DSMZ—Deutsche Sammlung von Mikroorganismen und Zellkulturen GmbH (DSMZ) (Inhoffenstr. 7 B, 38124 Braunschweig, Germany), recognized as an International Depositary Authority by the Budapest Treaty and the World Intellectual Property organization. Original deposit has been made on 13 Dec. 2022.

Purified cultures of the microbial strains Veillonella atypica LMG P-32913 (also denoted as Veillonella atypica PD221), Agathobacter rectalis LMG P-32914 (also denoted as Agathobacter rectalis PD406), and Agathobacter rectalis LMG P-32915 (also denoted as Agathobacter rectalis PD408) described in the present application were deposited by depositor MRM Health N.V. (Technologiepark 82, 9052 Gent, Belgium; physical address: Technologiepark 73, 9052 Zwijnaarde, Belgium) with the Belgian Coordinated Collections of Microorganisms (BCCM) (BCCM/LMG Bacteria collection, Universiteit Gent, Laboratorium voor Microbiologie, K. L. Ledeganckstraat 35, 9000 Gent, Belgium), recognized as an International Depositary Authority by the Budapest Treaty and the World Intellectual Property organization. Original deposits have been made on 9 Dec. 2022.

Purified cultures of the microbial strain Phascolarctobacterium faecium LMG P-32922 (also denoted as Phascolarctobacterium faecium RCC1614) described in the present application were deposited by depositor VIB VZW (Rijvisschestraat 120, 9052 Gent, Belgium; current address: Suzanne Tassierstraat 1, 9052 Gent, Belgium) with the Belgian Coordinated Collections of Microorganisms (BCCM) (BCCM/LMG Bacteria collection, Universiteit Gent, Laboratorium voor Microbiologie, K. L. Ledeganckstraat 35, 9000 Gent, Belgium), recognized as an International Depositary Authority by the Budapest Treaty and the World Intellectual Property organization. Original deposits have been made on 15 Dec. 2022.

### DESCRIPTION

### Compositions and Strains

An aspect of this disclosure relates to a strain of Phascolarctobacterium faecium having a 16S rRNA or rDNA sequence showing at least 97% sequence identity to the 16S rRNA or rDNA sequence of the Phascolarctobacterium faecium strain having accession number LMG P-32922 (SEQ ID NO: 1). A preferred aspect of this disclosure relates to a strain of Phascolarctobacterium faecium having accession number LMG P-32922 (also denoted as Phascolarctobacterium faecium RCC1614). Compositions comprising such strains are also encompassed.

An aspect of this disclosure relates to a strain of Veillonella atypica having a 16S rRNA or rDNA sequence showing at least 97% sequence identity to the 16S rRNA or rDNA sequence of the Veillonella atypica strain having accession number LMG P-32913 (SEQ ID NO: 2). A preferred aspect of this disclosure relates to a strain of Veillonella atypica having accession number LMG P-32913 (also denoted as Veillonella atypica PD221). Compositions comprising such strains are also encompassed.

An aspect of this disclosure relates to a strain of Agathobacter rectalis having a 16S rRNA or rDNA sequence showing at least 97% sequence identity to the 16S rRNA or rDNA sequence of the Agathobacter rectalis strain having accession number DSM 34506 (SEQ ID NO: 3). A preferred aspect of this disclosure relates to a strain of Agathobacter rectalis having accession number DSM 34506 (also denoted as Agathobacter rectalis DGCC14098). Compositions comprising such strains are also encompassed.

An aspect of this disclosure relates to a strain of Agathobacter rectalis having a 16S rRNA or rDNA sequence showing at least 97% sequence identity to the 16S rRNA or rDNA sequence of the Agathobacter rectalis strain having accession number LMG P-32914 (SEQ ID NO: 6). A preferred aspect of this disclosure relates to a strain of Agathobacter rectalis having accession number LMG P-32914 (also denoted as Agathobacter rectalis PD406). Compositions comprising such strains are also encompassed.

An aspect of this disclosure relates to a strain of Agathobacter rectalis having a 16S rRNA or rDNA sequence showing at least 97% sequence identity to the 16S rRNA or rDNA sequence of the Agathobacter rectalis strain having accession number LMG P-32915 (SEQ ID NO: 7). A preferred aspect of this disclosure relates to a strain of Agathobacter rectalis having accession number LMG P-32915 (also denoted as Agathobacter rectalis PD408). Compositions comprising such strains are also encompassed.

In some embodiments, the above-mentioned strains having 16S rRNA or rDNA sequences showing at least 97% sequence identity to a particular 16S rRNA or rDNA sequence (such as SEQ ID NOs: 1-7) may show 97.5%, 98%, 98.5%, 99%, 99.5%, 99.7%, 99.9%, or 100% sequence identity to said 16S rRNA or rDNA (such as SEQ ID NOs: 1-7).

In an aspect, there is provided a composition of two or more bacterial strains, wherein the composition comprises a bacterial strain of at least two of the following genera: Phascolarctobacterium, Veillonella, Eubacterium, Agathobacter, Lactiplantibacillus, and Anaerostipes. In other words, this means that the composition comprises minimally two bacterial strains, both from a different genus selected from Phascolarctobacterium, Veillonella, Eubacterium, Agathobacter, Lactiplantibacillus, and Anaerostipes. Thus, for example, a composition according to this disclosure that comprises three bacterial strains may comprise two strains from one genus and a third strain from a different genus, or alternatively, three strains from a different genus. In preferred embodiments, such compositions may comprise (at least) a bacterial strain of the genus Veillonella and a bacterial strain of the genus Anaerostipes.

In some embodiments, there is provided a composition of two or more bacterial strains, wherein the composition comprises a bacterial strain of at least two of the following genera: Phascolarctobacterium, Veillonella, Agathobacter, Lactiplantibacillus, and Anaerostipes. In preferred embodiments, such compositions may comprise (at least) a bacterial strain of the genus Veillonella and a bacterial strain of the genus Anaerostipes.

In some embodiments, there is provided a composition of two or more bacterial strains, wherein the composition comprises a bacterial strain of at least two of the following genera: Phascolarctobacterium, Veillonella, Agathobacter, and Anaerostipes.

In some embodiments, there is provided a composition of two or more bacterial strains, wherein the composition comprises a bacterial strain of at least two of the following genera: Phascolarctobacterium, Anaerostipes and Veillonella. In some embodiments, there is provided a composition of two or more bacterial strains, wherein the composition comprises a bacterial strain of at least two of the following genera: Agathobacter, Anaerostipes and Veillonella.

In some embodiments, there is provided a composition of two or more bacterial strains, wherein the composition comprises a bacterial strain of at least two of the following genera: Anaerostipes and Veillonella.

Preferably, compositions (and strains) described herein are for use in medicine. Preferred medical uses and methods are described in more detail elsewhere herein (see section “methods and uses”).

The genus Lactiplantibacillus was previously included in the genus Lactobacillus (as described in Zheng et al. Int J Syst Evol Microbiol 2020; 70(4):2782-2858).

A bacterial strain, as used herein, may be understood to refer to a genetic variant or subtype of a bacterium. Several different strains can belong to the same bacterial species. Throughout this disclosure, the term “bacterial strain” can also be replaced with the term “bacterium” or “bacterial member” and the like. In any of the compositions provided herein, the bacterial strains may be isolated or purified, for example, from a source such as a culture or a microbiota sample (e.g., fecal matter). Thus, throughout this disclosure, the term “bacterial strain” can also be replaced with the terms “purified bacterial strain” and “isolated bacterial strain”. As used herein, the terms “isolated” and “purified” refers to a bacterium or bacterial strain or bacterial member or a composition thereof that has been separated from one or more associated substances found in a source material or any material associated with the bacterium in any process used to produce the preparation, such as another bacterium or bacterial strain, one or more components of a growth medium, and/or one or more components of a sample, such as a fecal sample. In some embodiments, the bacteria are “substantially isolated” or “substantially purified”, for example from a source, such that other components of the source are not detected.

Compositions provided herein may comprise any suitable number of bacterial strains. In some embodiments, the number of bacterial strains comprised in the compositions of this disclosure is from 1 to 15, preferably from 2 to 15, more preferably from 2 to 12, even more preferably from 3 to 10, most preferably from 4 to 8. In some embodiments, the number of bacterial strains is at least two, preferably at least three, more preferably at least four. In some embodiments, the number of bacterial strains is four. In some embodiments, the number of bacterial strains is five. In some embodiments, the number of bacterial strains is six.

If the number of bacterial strains is 2, such a composition preferably includes an Anaerostipes and a Veillonella strain. If the number of bacterial strains is 3, such a composition preferably includes an Anaerostipes, a Veillonella, and a Phascolarctobacterium strain or an Anaerostipes, a Veillonella, and an Agathobacter strain. If the number of bacterial strains is 4, such a composition preferably includes an Anaerostipes, a Veillonella, a Phascolarctobacterium and an Agathobacter strain.

Any of the bacterial strains comprised in the compositions described herein are, preferably, commensal bacteria such as commensal gut bacteria. Accordingly, any of the bacterial strains comprised in the compositions described herein may be isolated or purified from a human subject, e.g. isolated or purified from fecal matter of a human subject. Any of the bacterial strains comprised in the compositions described herein are preferably non-pathogenic.

In some embodiments, a composition as described herein comprises a bacterial strain of at least three or at least four of the following genera: Phascolarctobacterium, Veillonella, Eubacterium, Agathobacter, Lactiplantibacillus, and Anaerostipes. In some embodiments, a composition as described herein comprises a bacterial strain of at least three or at least four of the following genera: Phascolarctobacterium, Veillonella, Agathobacter, Lactiplantibacillus, and Anaerostipes. In preferred embodiments, such compositions may comprise (at least) a bacterial strain of the genus Veillonella, a bacterial strain of the genus Anaerostipes, and a bacterial strain of the genus Phascolarctobacterium, optionally further comprising a bacterial strain of the genus Agathobacter or a bacterial strain of the genus Lactiplantibacillus, preferably a bacterial strain of the genus Agathobacter.

In some embodiments, a composition as described herein comprises a bacterial strain of at least three or at least four of the following genera: Phascolarctobacterium, Veillonella, Agathobacter, and Anaerostipes. In some embodiments, a composition as described herein comprises three or more, or four or more, bacterial strains of at least three resp. at least four of the following genera: Phascolarctobacterium, Veillonella, Eubacterium, Agathobacter, Lactiplantibacillus, and Anaerostipes. In some embodiments, a composition as described herein comprises three or more, or four or more, bacterial strains of at least three resp. at least four of the following genera: Phascolarctobacterium, Veillonella, Agathobacter, Lactiplantibacillus, and Anaerostipes. In some embodiments, a composition as described herein comprises three or more, or four or more, bacterial strains of at least three resp. at least four of the following genera: Phascolarctobacterium, Veillonella, Agathobacter, and Anaerostipes.

In some embodiments, a composition as described herein comprises a bacterial strain of at least five of the following genera: Phascolarctobacterium, Veillonella, Eubacterium, Agathobacter, Lactiplantibacillus, and Anaerostipes. In some embodiments, a composition as described herein comprises a bacterial strain of each of the following genera: Phascolarctobacterium, Veillonella, Agathobacter, Lactiplantibacillus, and Anaerostipes.

In some embodiments, a composition as described herein comprises a bacterial strain of each of the following genera: Phascolarctobacterium, Veillonella, Eubacterium, Agathobacter, Lactiplantibacillus, and Anaerostipes. In some embodiments, a composition as described herein comprises a bacterial strain of each of the following genera: Phascolarctobacterium, Veillonella, Agathobacter, and Anaerostipes.

In some embodiments, there is provided a composition comprising six or more bacterial strains of the following genera: Phascolarctobacterium, Veillonella, Eubacterium, Agathobacter, Lactiplantibacillus, and Anaerostipes. Such compositions thus comprise at least one bacterial strain of each of said six genera. In some embodiments, a composition may comprise or consist essentially of one strain of each of said six genera. In a preferred embodiment, there is provided a composition comprising five or more bacterial strains of the following genera: Phascolarctobacterium, Veillonella, Agathobacter, Lactiplantibacillus, and Anaerostipes. Such compositions thus comprise at least one bacterial strain of each of said five genera. In some embodiments, a composition may comprise or consist essentially of one strain of each of said five genera.

As described above, compositions provided herein may comprise two, three, four, five, six, or more bacterial strains belonging to the genera Phascolarctobacterium, Veillonella, Eubacterium, Agathobacter, Lactiplantibacillus, and Anaerostipes. In some embodiments, the two, three, four, five, six, or more bacterial strains are selected from particular species within said genera.

A preferred species of Phascolarctobacterium is Phascolarctobacterium faecium.

A preferred species of Veillonella is Veillonella atypica.

A preferred species of Agathobacter is Agathobacter rectalis.

A preferred species of Lactiplantibacillus is Lactiplantibacillus plantarum.

A preferred species of Anaerostipes is Anaerostipes caccae.

An example of a suitable Eubacterium species is Eubacterium limosum.

Agathobacter rectalis is also known as Eubacterium rectale (Rosero et al. Int J Syst Evol Microbiol. 2016; 66:768-773).

The above-described species are well-known to a skilled person. In particular, the bacterial species Phascolarctobacterium faecium (Del Dot et al. Phascolarctobacterium faecium gen. nov., spec. nov., a novel taxon of the Sporomusa group of bacteria. Syst Appl Microbiol (1993) 16:380-384), Veillonella atypica (Mays et al. Taxonomy of the genus Veillonella Prevot. Int J Syst Bacteriol 1982; 32:28-36), Agathobacter rectalis (Rosero et al. Int J Syst Evol Microbiol. 2016; 66:768-773; Mukherjee et al. Gut microbes from the phylogenetically diverse genus Eubacterium and their various contributions to gut health. Gut Microbes 2020; 12(1):1802866), Lactiplantibacillus plantarum (Walter. Ecological Role of Lactobacilli in the Gastrointestinal Tract: Implications for Fundamental and Biomedical Research. Appl Environ Microbiol 2008; 74:4985-4996; Zheng et al. Int J Syst Evol Microbiol 2020; 70(4):2782-2858) and Anaerostipes caccae (Schwiertz et al. Anaerostipes caccae gen. nov., sp. nov., a new saccharolytic, acetate-utilising, butyrate-producing bacterium from human faeces. Syst Appl Microbiol 2002; 25:46-51) are well-known bacterial species to a skilled person. The species Lactiplantibacillus plantarum was previously known as Lactobacillus plantarum (Zheng et al. Int J Syst Evol Microbiol 2020; 70(4):2782-2858).

Accordingly, in some embodiments, there is provided a composition of two or more bacterial strains, said composition comprising a bacterial strain of at least two of the following species: Phascolarctobacterium faecium, Veillonella atypica, Agathobacter rectalis, Lactiplantibacillus plantarum, and Anaerostipes caccae. In other words, this means that the composition comprises minimally two bacterial strains, both from a different species selected from Phascolarctobacterium faecium, Veillonella atypica, Agathobacter rectalis, Lactiplantibacillus plantarum, and Anaerostipes caccae. Thus, for example, such a composition according to this disclosure that comprises three bacterial strains may comprise two strains from one species and a third strain from a different species, or alternatively, three strains from a different species. In preferred embodiments, such compositions may comprise (at least) a bacterial strain of the species Veillonella atypica and a bacterial strain of the species Anaerostipes caccae.

In some embodiments, there is provided a composition of two or more bacterial strains, said composition comprising a bacterial strain of at least two of the following species: Phascolarctobacterium faecium, Veillonella atypica, Eubacterium sp., Agathobacter rectalis, Lactiplantibacillus plantarum, and Anaerostipes caccae.

In some embodiments, there is provided a composition of two or more bacterial strains, said composition comprising a bacterial strain of at least two of the following species: Phascolarctobacterium faecium, Veillonella atypica, Agathobacter rectalis, and Anaerostipes caccae.

In some embodiments, there is provided a composition of two or more bacterial strains, wherein the composition comprises a bacterial strain of at least two of the following species: Phascolarctobacterium faecium, Anaerostipes caccae and Veillonella atypica. In some embodiments, there is provided a composition of two or more bacterial strains, wherein the composition comprises a bacterial strain of at least two of the following species: Agathobacter rectalis, Anaerostipes caccae and Veillonella atypica.

In some embodiments, there is provided a composition of two or more bacterial strains, wherein the composition comprises a bacterial strain of at least two of the following species: Anaerostipes caccae and Veillonella atypica.

In some embodiments, a composition as described herein comprises a bacterial strain of at least three or at least four of the following species: Phascolarctobacterium faecium, Veillonella atypica, Agathobacter rectalis, Lactiplantibacillus plantarum, and Anaerostipes caccae. In preferred embodiments, such compositions may comprise (at least) a bacterial strain of the species Veillonella, a bacterial strain of the species Anaerostipes caccae, and a bacterial strain of the species Phascolarctobacterium faecium, optionally further comprising a bacterial strain of the species Agathobacter rectalis or a bacterial strain of the species Lactiplantibacillus plantarum, preferably a bacterial strain of the species Agathobacter rectalis.

In some embodiments, a composition as described herein comprises a bacterial strain of at least three or at least four of the following species: Phascolarctobacterium faecium, Veillonella atypica, Agathobacter rectalis, and Anaerostipes caccae.

In some embodiments, a composition as described herein comprises three or more, or four or more, bacterial strains of at least three resp. at least four of the following species: Phascolarctobacterium faecium, Veillonella atypica, Agathobacter rectalis, Lactiplantibacillus plantarum, and Anaerostipes caccae. In preferred embodiments, such compositions may comprise (at least) a bacterial strain of the species Veillonella atypica, a bacterial strain of the species Anaerostipes caccae, and a bacterial strain of the species Phascolarctobacterium faecium, optionally further comprising a bacterial strain of the species Agathobacter rectalis or a bacterial strain of the species Lactiplantibacillus plantarum, preferably a bacterial strain of the species Agathobacter rectalis.

In some embodiments, a composition as described herein comprises three or more, or four or more, bacterial strains of at least three resp. at least four of the following species: Phascolarctobacterium faecium, Veillonella atypica, Agathobacter rectalis, and Anaerostipes caccae.

In some embodiments, a composition as described herein comprises a bacterial strain of at least three or at least four of the following species: Phascolarctobacterium faecium, Veillonella atypica, Eubacterium sp., Agathobacter rectalis, Lactiplantibacillus plantarum, and Anaerostipes caccae. In some embodiments, a composition as described herein comprises three or more, or four or more, bacterial strains of at least three resp. at least four of the following species: Phascolarctobacterium faecium, Veillonella atypica, Eubacterium sp., Agathobacter rectalis, Lactiplantibacillus plantarum, and Anaerostipes caccae.

In some embodiments, a composition as described herein comprises a bacterial strain of each of the following species: Phascolarctobacterium faecium, Veillonella atypica, Agathobacter rectalis, Lactiplantibacillus plantarum, and Anaerostipes caccae. In some embodiments, a composition as described herein comprises a bacterial strain of each of the following species: Phascolarctobacterium faecium, Veillonella atypica, Agathobacter rectalis, and Anaerostipes caccae.

In some embodiments, a composition as described herein comprises a bacterial strain of at least five of the following species: Phascolarctobacterium faecium, Veillonella atypica, Eubacterium sp., Agathobacter rectalis, Lactiplantibacillus plantarum, and Anaerostipes caccae.

In some embodiments, a composition as described herein comprises a bacterial strain of each of the following species: Phascolarctobacterium faecium, Veillonella atypica, Eubacterium sp., Agathobacter rectalis, Lactiplantibacillus plantarum, and Anaerostipes caccae.

In a preferred embodiment, there is provided a composition comprising five or more bacterial strains of the following species: Phascolarctobacterium faecium, Veillonella atypica, Agathobacter rectalis, Lactiplantibacillus plantarum, and Anaerostipes caccae. Such compositions thus comprise at least one bacterial strain of each of said five species. In some embodiments, a composition may comprise or consist essentially of one strain of each of said five species. In another preferred embodiment, there is provided a composition comprising four or more bacterial strains of the following species: Phascolarctobacterium faecium, Veillonella atypica, Agathobacter rectalis, and Anaerostipes caccae. Such compositions thus comprise at least one bacterial strain of each of said four species. In some embodiments, a composition may comprise or consist essentially of one strain of each of said four species.

In some embodiments, there is provided a composition comprising six or more bacterial strains of the following species: Phascolarctobacterium faecium, Veillonella atypica, Eubacterium sp., Agathobacter rectalis, Lactiplantibacillus plantarum, and Anaerostipes caccae. Such compositions thus comprise at least one bacterial strain of each of said six species. In some embodiments, a composition may comprise or consist essentially of one strain of each of said six species.

As described above, compositions provided herein may comprise two, three, four, five, six, or more bacterial strains belonging to the species Phascolarctobacterium faecium, Veillonella atypica, Eubacterium sp., Agathobacter rectalis, Lactiplantibacillus plantarum, and Anaerostipes caccae. In some embodiments, the two, three, four, five, six, or more bacterial strains are selected from particular strains within said species.

Preferred strains of Phascolarctobacterium faecium are Phascolarctobacterium faecium LMG P-32922, and strains having 16S rRNA or rDNA sequences showing at least 97% sequence identity to the 16S rRNA or rDNA sequence of said strain (SEQ ID NO: 1). Phascolarctobacterium faecium LMG P-32922 is preferred. Preferred strains of Veillonella atypica are Veillonella atypica LMG P-32913, and strains having 16S rRNA or rDNA sequences showing at least 97% sequence identity to the 16S rRNA or rDNA sequence of said strain (SEQ ID NO: 2). Veillonella atypica LMG P-32913 is preferred.

Preferred strains of Agathobacter rectalis are Agathobacter rectalis DSM 34506, Agathobacter rectalis LMG P-32914, Agathobacter rectalis LMG P-32915, and strains having 16S rRNA or rDNA sequences showing at least 97% sequence identity to the 16S rRNA or rDNA sequence of Agathobacter rectalis DSM 34506 (SEQ ID NO: 3), Agathobacter rectalis LMG P-32914 (SEQ ID NO: 6), or Agathobacter rectalis LMG P-32915 (SEQ ID NO: 7). More preferred strains of Agathobacter rectalis are Agathobacter rectalis DSM 34506, and strains having 16S rRNA or rDNA sequences showing at least 97% sequence identity to the 16S rRNA or rDNA sequence of said strain (SEQ ID NO: 3). Agathobacter rectalis DSM 34506 is preferred.

Preferred strains of Lactiplantibacillus plantarum are Lactiplantibacillus plantarum LMG P-29366, and strains having 16S rRNA or rDNA sequences showing at least 97% sequence identity to the 16S rRNA or rDNA sequence of said strain (SEQ ID NO: 4). Lactiplantibacillus plantarum LMG P-29366 is preferred.

Preferred strains of Anaerostipes caccae are Anaerostipes caccae LMG P-29359, and strains having 16S rRNA or rDNA sequences showing at least 97% sequence identity to the 16S rRNA or rDNA sequence of said strain (SEQ ID NO: 5). Anaerostipes caccae LMG P-29359 is preferred.

In some embodiments, the above-mentioned strains having 16S rRNA or rDNA sequences showing at least 97% sequence identity to a particular 16S rRNA or rDNA sequence (such as SEQ ID NOs: 1-7) may show 97.5%, 98%, 98.5%, 99%, 99.5%, 99.7%, 99.9%, or 100% sequence identity to said 16S rRNA or rDNA (such as SEQ ID NOs: 1-7).

As used herein, the term “16S ribosomal RNA” or “16S rRNA” refers to a nucleic acid sequence which is a component of the small prokaryotic ribosomal subunit (30S). The 16S rRNA is known to act as a scaffold defining the positions of the ribosomal proteins. The genes coding for it are referred to as 16S rRNA genes or 16S rDNA and are commonly used for phylogenetic studies, as they are known to be highly conserved sequences. It is well known to a person skilled in the art that 16S rRNA or rDNA sequences can be deposited in online sequence databases, for example at ncbi's GenBank (https://www.ncbi.nlm.nih.gov/genbank/) and that they can be retrieved based on their unique accession number for use as reference 16S rRNA or rDNA sequence in evaluation of sequence homology, as for example described by Eeckhaut et al. Int J Syst Evol Microbiol 2008; 58:2799-2802.

In some embodiments, a composition as described herein comprises two or more bacterial strains selected from the group consisting of Phascolarctobacterium faecium LMG P-32922, Veillonella atypica LMG P-32913, Agathobacter rectalis DSM 34506, Lactiplantibacillus plantarum LMG P-29366 and Anaerostipes caccae LMG P-29359. Agathobacter rectalis DSM 34506 may alternatively be replaced with Agathobacter rectalis LMG P-32914 or Agathobacter rectalis LMG P-32915. In preferred embodiments, such compositions may comprise (at least) Veillonella atypica LMG P-32913 and Anaerostipes caccae LMG P-29359.

In some embodiments, a composition as described herein comprises two or more bacterial strains selected from the group consisting of Phascolarctobacterium faecium LMG P-32922, Veillonella atypica LMG P-32913, Agathobacter rectalis DSM 34506, and Anaerostipes caccae LMG P-29359. Agathobacter rectalis DSM 34506 may alternatively be replaced with Agathobacter rectalis LMG P-32914 or Agathobacter rectalis LMG P-32915.

In some embodiments, a composition as described herein comprises two or more bacterial strains selected from the group consisting of Phascolarctobacterium faecium LMG P-32922, Veillonella atypica LMG P-32913, and Anaerostipes caccae LMG P-29359.

In some embodiments, a composition as described herein comprises two or more bacterial strains selected from the group consisting of Veillonella atypica LMG P-32913, Agathobacter rectalis DSM 34506, and Anaerostipes caccae LMG P-29359. Agathobacter rectalis DSM 34506 may alternatively be replaced with Agathobacter rectalis LMG P-32914 or Agathobacter rectalis LMG P-32915.

In some embodiments, a composition as described herein comprises two or more bacterial strains selected from the group consisting of Veillonella atypica LMG P-32913 and Anaerostipes caccae LMG P-29359.

In some embodiments, a composition as described herein comprises three or more, or four or more, bacterial strains selected from the group consisting of Phascolarctobacterium faecium LMG P-32922, Veillonella atypica LMG P-32913, Agathobacter rectalis DSM 34506, Lactiplantibacillus plantarum LMG P-29366 and Anaerostipes caccae LMG P-29359. Agathobacter rectalis DSM 34506 may alternatively be replaced with Agathobacter rectalis LMG P-32914 or Agathobacter rectalis LMG P-32915. In preferred embodiments, such compositions may comprise (at least) Veillonella atypica LMG P-32913 Anaerostipes caccae LMG P-29359, and Phascolarctobacterium faecium LMG P-32922, optionally further comprising Agathobacter rectalis DSM 34506 or Lactiplantibacillus plantarum LMG P-29366, preferably Agathobacter rectalis DSM 34506.

In some embodiments, a composition as described herein comprises three or more, or four or more, bacterial strains selected from the group consisting of Phascolarctobacterium faecium LMG P-32922, Veillonella atypica LMG P-32913, Agathobacter rectalis DSM 34506, and Anaerostipes caccae LMG P-29359. Agathobacter rectalis DSM 34506 may alternatively be replaced with Agathobacter rectalis LMG P-32914 or Agathobacter rectalis LMG P-32915.

In some embodiments, a composition as described herein comprises Phascolarctobacterium faecium LMG P-32922, Veillonella atypica LMG P-32913, Agathobacter rectalis DSM 34506, Lactiplantibacillus plantarum LMG P-29366 and Anaerostipes caccae LMG P-29359. Agathobacter rectalis DSM 34506 may alternatively be replaced with Agathobacter rectalis LMG P-32914 or Agathobacter rectalis LMG P-32915.

In some embodiments, a composition as described herein comprises Phascolarctobacterium faecium LMG P-32922, Veillonella atypica LMG P-32913, Agathobacter rectalis DSM 34506, and Anaerostipes caccae LMG P-29359. Agathobacter rectalis DSM 34506 may alternatively be replaced with Agathobacter rectalis LMG P-32914 or Agathobacter rectalis LMG P-32915.

In some embodiments, a composition as described herein comprises Veillonella atypica LMG P-32913, Agathobacter rectalis DSM 34506, and Anaerostipes caccae LMG P-29359. Agathobacter rectalis DSM 34506 may alternatively be replaced with Agathobacter rectalis LMG P-32914 or Agathobacter rectalis LMG P-32915.

In some embodiments, a composition as described herein comprises Phascolarctobacterium faecium LMG P-32922, Veillonella atypica LMG P-32913, and Anaerostipes caccae LMG P-29359.

In some embodiments, a composition as described herein comprises Veillonella atypica LMG P-32913 and Anaerostipes caccae LMG P-29359.

In some embodiments, a composition as described herein consists essentially of Phascolarctobacterium faecium LMG P-32922, Veillonella atypica LMG P-32913, Agathobacter rectalis DSM 34506, Lactiplantibacillus plantarum LMG P-29366 and Anaerostipes caccae LMG P-29359. Agathobacter rectalis DSM 34506 may alternatively be replaced with Agathobacter rectalis LMG P-32914 or Agathobacter rectalis LMG P-32915.

In some embodiments, a composition as described herein consists essentially of Phascolarctobacterium faecium LMG P-32922, Veillonella atypica LMG P-32913, Agathobacter rectalis DSM 34506, and Anaerostipes caccae LMG P-29359. Agathobacter rectalis DSM 34506 may alternatively be replaced with Agathobacter rectalis LMG P-32914 or Agathobacter rectalis LMG P-32915.

In some embodiments, a composition as described herein consists essentially of Veillonella atypica LMG P-32913, Agathobacter rectalis DSM 34506, and Anaerostipes caccae LMG P-29359. Agathobacter rectalis DSM 34506 may alternatively be replaced with Agathobacter rectalis LMG P-32914 or Agathobacter rectalis LMG P-32915.

In some embodiments, a composition as described herein consists essentially of Phascolarctobacterium faecium LMG P-32922, Veillonella atypica LMG P-32913, and Anaerostipes caccae LMG P-29359.

In some embodiments, a composition as described herein consists essentially of Veillonella atypica LMG P-32913 and Anaerostipes caccae LMG P-29359.

Further provided herein are compositions as described elsewhere, wherein additional bacterial strains are added. Accordingly, in some embodiments, a composition as described herein further comprises one or more additional bacterial strains.

In some embodiments, a composition as described herein further comprises a bacterial strain from the genus Faecalibacterium, preferably from the species Faecalibacterium prausnitzii or Faecalibacterium duncaniae, preferably Faecalibacterium duncaniae.

In some embodiments, a composition as described herein further comprises one or more bacterial strains from the genera Bifidobacterium, Oscillibacter, Dysosmobacter or Eubacterium.

In some embodiments, a composition as described herein further comprises one or more bacterial strains from the genera Faecalibacterium (preferably from the species Faecalibacterium prausnitzii or Faecalibacterium duncaniae), Bifidobacterium, Oscillibacter, Dysosmobacter or Eubacterium.

The genus Eubacterium, as used herein, is understood to encompass bacteria characterized by producing mixtures of organic acids from carbohydrates or peptone, which may include copious amounts of butyric, acetic and formic acids but do not produce: (a) only lactic acid, (b) propionic acid as the major acid, (c) greater quantities of acetic acid than lactic acid with or without the formation of formic acid and (d) lactic and succinic acid with small quantities of acetic or formic acid (Wade W G. The genus Eubacterium and related genera. Prokaryotes. 2006; 4:823-835). Thus, the genus Eubacterium as used herein is not limited to the genus sensu stricto but refers to Eubacterium as a combined group—Eubacterium et rel. (Mukherjee et al. Gut microbes from the phylogenetically diverse genus Eubacterium and their various contributions to gut health. Gut Microbes. 2020 Nov. 9; 12(1):1802866).

### Further Components

In a further aspect, the compositions described herein may further comprise one or more prebiotics. The term ‘prebiotic’ refers to any chemical that is capable of inducing or promoting the growth or activity of microorganisms (e.g., bacteria) that contribute to the well-being of their host. Hence, prebiotics can influence or alter the composition of organisms in the gut microbiome. However, in principle it is a more general term that can refer to other areas of the body as well. Typically—but non-limiting—prebiotics are non-digestible fiber compounds that at least partially pass undigested through the upper part of the gastrointestinal tract and stimulate the growth or activity of advantageous bacteria that colonize the large bowel by acting as substrate for them.

In some embodiments, a composition as described herein may comprise a bacterial strain capable of producing prebiotic metabolites.

In a further aspect, the compositions described herein may further comprise one or more therapeutic agents. The term ‘agent’ as used herein may refer to a chemical compound, a small molecule, a mixture of chemical compounds and/or a biological macromolecule (such as a nucleic acid, an antibody, an antibody fragment, a protein or peptide). The activity of such agents may render them suitable as a “therapeutic agent” which is a biologically, physiologically, or pharmacologically active substance (or substances) that acts locally or systemically in a subject.

In some embodiments, said therapeutic agent is selected from the group consisting of non-steroidal anti-inflammatory drugs (NSAIDs), glucocorticoids, and disease-modifying antirheumatic drugs (DMARDs). Preferred therapeutic agents are DMARDs. Therapeutic agents belonging to the NSAIDs, steroid drugs and DMARDs are well-known in the art and have been described extensively, see e.g. Radu & Bungeau. Management of Rheumatoid Arthritis: An Overview. Cells 2021; 10(11): 2857, incorporated herein by reference in its entirety.

Suitable examples of NSAIDs include naproxen, ibuprofen and coxibs (COX-2 selective inhibitors).

Suitable examples of glucocorticoids include prednisone, hydrocortisone, prednisolone and dexamethasone.

Suitable examples of DMARDs may belong to the conventional synthetic DMARDs (csDMARDs), biologic DMARDs (bDMARDs) or targeted synthetic DMARDs (tsDMARDs). Preferred DMARDs are bDMARDs. Suitable examples of bDMARDs include
TNF-alpha inhibitors (e.g. etanercept, infliximab, golimumab, adalimumab, certolizumab pegol);
B-cell depleters (e.g. rituximab, ofatumumab);
B-cell receptor inhibitors (e.g. belimumab, atacicept, tabalumab);
Antagonists of CD28 on T-cells (e.g. abatacept, belatacept);
IL-1 inhibitors (e.g. anakinra, canakinumab, rilonacept);
IL-6 inhibitors (e.g. tocilizumab, sarilumab, sirukumab, olokizumab, clazakizumab);
IL 12/23 inhibitors (e.g. ustekinumab);
IL-17 inhibitors (e.g. ixekizumab, secukinumab, brodalumab);
Granulocyte-macrophage colony-stimulating factor inhibitors (e.g. mavrilimumab, otilimab); and
RANKL inhibitors (e.g. denosumab).

TNF-alpha inhibitors (also named TNF inhibitors) are preferred bDMARDs.

In a further aspect, provided herein is a kit or a kit-of-parts comprising any of the compositions of this disclosure. In some embodiments, a kit as described herein comprises a composition of two or more bacterial strains as described herein, and a therapeutic agent as described herein (or a composition thereof).

In a further aspect, the compositions described herein may further comprise succinic acid, or a salt or ester thereof. Without wishing to be bound by theory, addition of succinic acid to the compositions disclosed herein may provide further advantages, e.g. in connection to reducing intestinal inflammation and inducing Tuft cell expansion.

Similarly, in a further aspect, the compositions described herein may further comprise one or more bacterial strains capable of producing succinic acid.

As used herein, a bacterial strain capable of succinic acid production may be understood to refer to a bacterial strain carrying at least one of the pathways for succinic acid production in its genome, such as the pathways described in “Fernández-Veledo et al. Gut microbiota-derived succinate: Friend or foe in human metabolic diseases?Rev Endocr Metab Disord 2019; 20:439-447”. Presence of these pathways in bacteria can be assessed by genetic methods known to the person skilled in the art. As an example, the presence of a marker gene can be detected by PCR or sequencing-based methods or using in silico phylogenetic tools. Alternatively, or additionally, succinic acid production can be assessed by chemical analysis of in vitro, in vivo or in situ samples using methods known to a person of skill in the art, including gas chromatography (GC), liquid chromatography (LC), nuclear magnetic resonance (NMR), and capillary electrophoresis (CE).

Main succinate producers in the gut include Bacteroides spp., Prevotella spp. and Veillonella spp. Accordingly, in some embodiments, the one or more bacterial strains capable of producing succinic acid belong to the genus Bacteroides, Prevotella and Veillonella. In some embodiments, the one or more bacterial strains capable of producing succinic acid belong to the species listed in Table 1 of “Fernández-Veledo et al. Gut microbiota-derived succinate: Friend or foe in human metabolic diseases?Rev Endocr Metab Disord 2019; 20:439-447” (Propionibacterium acidipropionici, Propionibacterium shermanii, Bacteroides fragilis, Alistipes indistinctus, Bacteroides vulgatus, Paraprevotella xylaniphila, Parabacteroides distasonis, Blautia wexlerae, Faecalibacterium prausnitzii, Ruminococcus albus, Citrobacter freundii, Succinivibrio dextrinosolvens, Akkermansia muciniphila).

### Specific Compositions and Derived Products

In some embodiments, the compositions described herein may be pharmaceutical compositions, i.e. a composition which further comprises pharmaceutically acceptable ingredients.

As used herein, “pharmaceutically acceptable ingredients” include pharmaceutically acceptable carriers, fillers, preservatives, solubilizers, vehicles, diluents and/or excipients. Accordingly, the one or more pharmaceutically acceptable ingredients may be selected from the group consisting of pharmaceutically acceptable carriers, fillers, preservatives, solubilizers, vehicles, diluents (e.g. starch, cellulose derivatives or sugar derivatives), and excipients, preferably selected from the group consisting of excipients, vehicles, carriers and diluents. Such pharmaceutically acceptable carriers, fillers, preservatives, solubilizers, vehicles, diluents and/or excipients may for instance be found in Remington: The Science and Practice of Pharmacy, 23rd edition. Elsevier (2020), incorporated herein by reference.

The compositions described herein may be formulated to provide a rapid and efficient therapeutic effect. In some embodiments, a composition as described herein may be formulated for delivery to the intestines, for example formulated as a composition for rectal administration or for oral administration, preferably for oral administration.

In some embodiments, the compositions described herein may be formulated as a capsule, microcapsule, tablet, granule, powder, troche, pill, food supplement, suspension, suppository, enema or syrup and the like, preferably a powder, a suspension, a tablet or a capsule. In some embodiments, the compositions as described herein may be provided in a suspension dosage form.

In some embodiments, the (pharmaceutical) compositions described herein may be formulated as a composition for rectal administration. In this regard, any of the compositions described herein can be provided as a (pharmaceutical) dosage form for rectal administration, such as a suppository or an enema.

In some embodiments, the (pharmaceutical) compositions described herein may be formulated as a composition for oral administration, such as a capsule, microcapsule, tablet, granule, powder, troche, pill, food supplement, suspension or syrup, preferably a powder, a suspension, a tablet or a capsule.

In some embodiments, the compositions described herein may comprise dried bacteria, e.g. lyophilized bacteria, or bacteria provided in an active culture format. In preferred embodiments, the bacteria may be lyophilized.

In some embodiments, particularly in the context of a composition comprising lyophilized bacteria, a composition as described herein may further comprise a lyoprotectant. A “lyoprotectant” as used herein protects the bacteria by preventing the damaging effects of lyophilization. An example of a lyoprotectant is serum.

In some embodiments, particularly in the context of a composition comprising bacteria provided in an active culture format, a composition as described herein may further comprise a growth medium. In some embodiments, the growth medium is a growth medium appropriate for the two or more bacterial strains as described herein. Non-limiting examples of such growth medium are unsupplemented OTEB (Oral Treponeme Enrichment Broth) or fastidious anaerobic agar (FAA) supplemented with 10% foetal bovine serum (FBS).

In some embodiments, a composition as described herein further comprises a buffer. Several suitable buffers are known to the skilled person (see Remington: The Science and Practice of Pharmacy, 23rd edition. Elsevier (2020), incorporated herein by reference), including for example a phosphate buffered saline solution.

In some embodiments, compositions described herein, particularly compositions comprising a growth medium as described herein, further comprise a cryoprotectant. A “cryoprotectant” as used herein protects the bacteria by preventing the damaging effects of water crystals when cells are frozen, more particularly at −60° C., or −70° C. or −80° C. or in liquid nitrogen. Non-limiting examples of a cryoprotectant are glycerol and trehalose.

In some embodiments, the compositions described herein can be incorporated in a food product, beverage, dietary supplement or nutraceutical. Aspects of the present disclosure thus also provide food products, beverages, dietary supplements and nutraceuticals comprising any of the compositions described herein. Also within the scope of the present disclosure are food products, beverages, dietary supplements and nutraceuticals comprising any (combinations) of the bacterial strains described herein.

A “food product” is typically an edible material composed primarily of one or more of the macronutrients protein, carbohydrate and fat. A food may also contain one or more micronutrients such as vitamins or minerals. Examples of foods in which the composition may be incorporated include snack bars, cereals, buns, muffins, biscuits, cakes, pastries, processed vegetables, sweets, probiotic formulations including yoghurts, beverages, plant oil-based liquids, animal fat-based liquids, frozen confections and cheeses. Preferred foods include yoghurts, cheeses and other dairy products.

Examples of beverages include soft beverages, syrups, squashes, dry drink mixes and nutritional beverages. A nutraceutical is a food ingredient, food supplement or food product which is considered to provide a medical or health benefit, including the prevention and treatment of disease. Nutraceuticals are sometimes also denoted as “functional food”, i.e. a food that is typically marketed as providing a health benefit beyond that of supplying pure nutrition to the consumer.

The disclosure also provides a probiotic comprising a composition discussed herein. In other words, in some embodiments, a composition as described herein is a probiotic composition. A probiotic is typically a live supplement which can enhance the intestinal microbiota. Such probiotics may be given in particularly to humans but also to farm and domestic animals and to aquatic organisms. The probiotic may additionally comprise one or more acceptable excipients or flavorings, which are suitable for ingestion by a human or animal.

In some embodiments, the compositions as described herein comprise at least 10\hat{ }2, preferably at least 10\hat{ }3, more preferably at least 10\hat{ }4, even more preferably at least 10\hat{ }5 bacteria. In some embodiments, the compositions as described herein comprise from 10\hat{ }2 to 10\hat{ }14, preferably from 10\hat{ }3 to 10\hat{ }13, more preferably from 10\hat{ }4 to 10\hat{ }12, even more preferably from 10\hat{ }5 to 10\hat{ }11 bacteria. In some embodiments, the compositions as described herein comprise at least 10\hat{ }5 bacteria, for example between 10\hat{ }5 and 10\hat{ }11 bacteria. In preferred embodiments, the number of bacteria means the number of live bacteria. In preferred embodiments, the number of live bacteria is expressed as the number of colony forming units (CFU).

It is understood that, with respect to any of the compositions provided throughout this disclosure, the term “comprising” may be replaced with the term “consisting essentially of” or with the term “consisting”. In other words, in some embodiments, the compositions provided herein consist essentially of the bacterial strains as provided, or consist of the bacterial strains as provided.

In addition to the compositions as described herein, a preferred aspect of this disclosure also relates to the same compositions for use in any of the methods described later herein (see section “methods and uses”).

### Methods for Obtaining Compositions; and Compositions Obtained Thereby

In some embodiments, the compositions as provided herein may be obtained by the methods described in this section. In some embodiments, the compositions as provided herein may be obtainable by the methods described in this section. In other words: in some embodiments, the bacterial strains comprised in the compositions provided herein are previously subjected to the methods described in this section. In this context, it can also be said that the bacterial strains are “preadapted” or “conditioned” by any of the methods described in this section.

The methods provided in this section impart certain advantageous characteristics on the compositions of this disclosure. In particular, the onset of the effect of the composition on a host will be quicker, and/or the effectiveness may be higher, i.e. they provide for a rapid and efficient therapeutic (and/or prophylactic) effect. More specifically, the “preadapted” or “conditioned” compositions may significantly decrease the time of biotherapeutic onset meaning that, by being preadapted, the set of microorganisms can exert their functionality at least 5% quicker (on a temporal scale), preferably at least 10% quicker, more preferably at least 20% quicker and most preferably at least 30% quicker as compared to a loosely assembled set of the same strains. The “preadapted” or “conditioned” compositions may also significantly increase the effect of treatment meaning that, by being preadapted, the set of microorganisms can exert their functionality with at least a 5% higher effectiveness, preferably at least 10% higher effectiveness, more preferably at least 20% and most preferably at least 30% higher effectiveness. The effectiveness depends on the endpoint for which the set of microorganisms has been designed. Possible functionalities include but are not limited to reduction of joint inflammation (joint inflammation is described elsewhere herein), grip strength, bone erosion and inflammatory infiltrates, among others.

In an aspect, there is provided a method for obtaining a composition as described herein, comprising growing the bacterial strains together in a reactor. In some embodiments, a method for obtaining a composition as described herein comprises the steps of:
culturing the bacterial strains together in a reactor; and
taking a sample from the reactor contents.

In some embodiments, a method for obtaining a composition as described herein comprises the steps of:
culturing the bacterial strains together in a reactor;
taking a sample from the reactor contents; and
further processing the sample to improve long-term viability of the bacterial strains.

In some embodiments, the step of growing the bacterial strains together in a reactor is performed for a certain time period, e.g. a period of from 1 to 15 days, preferably a period of from 3 to 10 days.

In some embodiments, further processing the sample to improve long-term viability of the bacterial strains includes cryopreservation or lyophilization, preferably lyophilization. Lyophilization can be performed directly on the reactor sample or after resuspension of the bacterial cells in a suitable lyophilization medium.

Accordingly, in some embodiments, a method for obtaining a composition as described herein comprises the steps of:
culturing the bacterial strains together in a reactor;
taking a sample from the reactor contents;
optionally, resuspending the bacterial cells in a suitable lyophilization medium; and
lyophilizing the resuspended bacterial cells.

In some embodiments, said reactor is maintained under continuous or semi-continuous conditions. In some embodiments, said reactor is maintained under standardized conditions representative for the gastro-intestinal (GI) tract.

The parameters characterizing the standardized conditions include but are not limited to: pH (range between 1.5 and 8); availability of carbon sources (either carbohydrate or proteins or a combination thereof); retention time in a specific reactor (range between 10 min and 200 h); oxygen availability (range between 0 and 8 g/L); availability of micronutrients; presence/absence of antibiotics; concentration of bile salts (range between 0 and 20 mM); presence of heavy metals; presence of host factors as immune molecules. In a preferred embodiment, the parameters characterizing the standardized conditions comprise pH, retention time in a specific reactor and concentration of bile salts, all as earlier defined herein. Depending on the complexity of the composition or “consortium”, a period of 1 to 15 days may be suitable to obtain a functionally stable consortium. On average, in order to develop a consortium composed of 7 to 14 members, a time between 3 and 10 days is suitable to obtain a functionally stable consortium (depending on the environmental conditions). A composition as defined herein is therefore obtainable after having been preadapted or cultured during a time between 1 and 15 days, preferably between 3 and 10 days, under conditions wherein pH, retention time in a specific reactor and concentration of bile salts have been set as defined herein. Such a process allows the production of a composition or consortium which is stable, including functionally stable.

In a further aspect, there is provided a reactor operating under standardized conditions representative for the GI tract, comprising: pH range between 1.5 and 8; availability of carbon sources; retention time between 10 min and 200 h; oxygen availability between 0 and 8 g/L; availability of micronutrients; presence/absence of antibiotics; concentration of bile salts between 0 and 20 mM; presence of heavy metals; presence of host factors as immune molecules. In an embodiment, said reactor is such that the parameters characterizing the standardized conditions comprise pH, retention time in a specific reactor and concentration of bile salts as defined in the previous paragraph. In an embodiment, such reactor comprises a composition of between 1 and 15, preferably between 2 and 15, more preferably between 2 and 12, even more preferably between 3 and 10, most preferably between 4 and 8 distinct bacteria members. In an embodiment, such reactor comprises a composition of two or more bacterial strains as described herein. In a preferred embodiment, such composition resides for a time between 1 and 15 days, preferably between 3 and 10 days, in such a reactor to obtain a (functionally) stable composition or consortium.

As used herein, “stable” or “functionally stable” composition or consortium may be a composition as described herein still comprising the initial different number of strains of bacteria after culturing the strains for a defined period of time. In other words, each of the strains comprised in a “stable” or “functionally stable” composition of the disclosure remains detectable for a defined period of time. In some embodiments, the majority of the strains comprised in a “stable” or “functionally stable” composition of the disclosure remain detectable for a defined period of time, wherein the majority of strains may mean all strains except one, or all strains except two.

In some embodiments, the total cell concentration of a “stable” or “functionally stable” composition or consortium, when cultured in a reactor, optionally under semi-continuous or continuous growth conditions, remains stable for a defined period of time after an initial adaptation phase. In some embodiments, an initial adaptation phase lasts 12 hours, 24 hours, 36 hours, 48 hours, 60 hours, or 72 hours.

In some embodiments, the cell concentration of each strain comprised in a “stable” or “functionally stable” composition or consortium, when cultured in a reactor, optionally under semi-continuous or continuous growth conditions, remains stable for a defined period of time after an initial adaptation phase. In some embodiments, an initial adaptation phase lasts 12 hours, 24 hours, 36 hours, 48 hours, 60 hours, or 72 hours.

In this context, “remaining stable” may mean that the variation in cell concentration remains within a range of +/−1 log unit, preferably +/−0.5 log unit. In some embodiments, particularly when considering individual strains, “remaining stable” may mean that the rank order of strains in terms of their abundance remains the same.

In some embodiments, the short chain fatty acid (SCFA) concentration in a reactor comprising a “stable” or “functionally stable” composition or consortium, optionally when cultured under semi-continuous or continuous growth conditions, remains stable for a defined period of time after an initial adaptation phase. In some embodiments, an initial adaptation phase lasts 12 hours, 24 hours, 36 hours, 48 hours, 60 hours, or 72 hours. In this context, “remaining stable” may mean that the variation in SCFA concentration remains within a range of +/−30%, preferably +/−15%.

SCFAs as used herein include formate (C1), acetate (C2), propionate (C3), butyrate (C4), and valerate (C5), preferably acetate, propionate, and butyrate, more preferably acetate and propionate.

In some embodiments, a “stable” or “functionally stable” composition may be characterized as follows, when cultured in a reactor, optionally under semi-continuous or continuous growth conditions:
the total cell concentration remains stable for a defined period of time after an initial adaptation phase;
the cell concentration of each strain remains stable for a defined period of time after an initial adaptation phase; and
the short chain fatty acid (SCFA) concentration remains stable for a defined period of time after an initial adaptation phase.

The defined period of time for assessing the (functional) stability of the compositions of this disclosure may be a period of at least three days, at least five days, at least seven days, or at least ten days.

Hence, within the context of compositions for use, methods and uses as described herein, the treatment may result in a faster biotherapeutic onset and/or increased effectiveness, i.e. rapid and efficient therapeutic effect, as described herein.

### Methods and Uses

As elaborated in the experimental part, it has been surprisingly found that the above-described compositions exert several direct and indirect beneficial effects, in particular on the joints and the intestines. Specifically, compositions as described herein exert at least the following beneficial effects: reduction of joint inflammation (joint inflammation is described elsewhere herein), increase in grip strength, reduction in bone erosion, and reduction in inflammatory infiltrates, among others. It therefore follows that the compositions provided herein have broad applicability in treating and preventing joint inflammation and diverse diseases and conditions associated therewith.

Accordingly, in a further aspect, the compositions as described herein are provided for use in medicine. In other words, the compositions as described herein are provided for use as a medicament.

Preferably, the compositions as described herein are for use in a method of treating or preventing joint inflammation. It is also encompassed herein to treat or prevent symptoms of joint inflammation and diseases or conditions underlying joint inflammation, each of which aspects are described in more detail elsewhere herein. Accordingly, in some embodiments, the compositions as described herein are provided for use in a method of treating or preventing joint inflammation or a symptom thereof. In some embodiments, the compositions as described herein are provided for use in a method of treating or preventing joint inflammation or an underlying disease or condition. In some embodiments, the compositions as described herein are provided for use in a method of treating or preventing joint inflammation, a symptom thereof, or an underlying disease or condition.

In a further aspect there is provided a method for treating or preventing joint inflammation, said method comprising administering a composition as described herein. In some embodiments there is provided a method for treating or preventing joint inflammation or a symptom thereof, said method comprising administering a composition as described herein. In some embodiments there is provided a method for treating or preventing joint inflammation or an underlying disease or condition, said method comprising administering a composition as described herein. In some embodiments there is provided a method for treating or preventing joint inflammation, a symptom thereof, or an underlying disease or condition, said method comprising administering a composition as described herein. In some embodiments, administering a composition means administering said composition to a subject, such as a subject in need thereof. In some embodiments, an effective amount or a therapeutically (and/or prophylactically) effective amount of a composition is administered.

In a further aspect there is provided a use of a composition as described herein, for the manufacture of a medicament for treating or preventing joint inflammation. In some embodiments there is provided a use of a composition as described herein, for the manufacture of a medicament for treating or preventing joint inflammation or a symptom thereof. In some embodiments there is provided a use of a composition as described herein, for the manufacture of a medicament for treating or preventing joint inflammation or an underlying disease or condition. In some embodiments there is provided a use of a composition as described herein, for the manufacture of a medicament for treating or preventing joint inflammation, a symptom thereof, or an underlying disease or condition.

In a further aspect, there is provided a use of a composition as described herein, for treating or preventing joint inflammation. In some embodiments there is provided a use of a composition as described herein, for treating or preventing joint inflammation or a symptom thereof. In some embodiments there is provided a use of a composition as described herein, for treating or preventing joint inflammation or an underlying disease or condition. In some embodiments there is provided a use of a composition as described herein, for treating or preventing joint inflammation, a symptom thereof, or an underlying disease or condition.

“Treating” or “treatment” as used herein may include delaying (e.g. delaying progression or delaying detoriation), ameliorating or curing. Accordingly, throughout this disclosure, “treating” and the like may be replaced with “treating, delaying, ameliorating or curing” and the like.

Within the context of compositions for use, methods and uses provided herein, “treating or preventing” as used herein may be understood to treat or prevent a disease, condition or symptom as compared to said disease, condition or symptom prior to administering the composition. In some embodiments, “treating or preventing” as used herein may be understood to treat or prevent a disease, condition or symptom as compared to said disease, condition or symptom in a subject inflicted with the same disease, condition or symptom receiving a placebo. Similarly, the occurrence of any of the effects described elsewhere herein may be compared in the same way.

Within the context of compositions for use, methods, and uses provided herein, an effective amount or a therapeutically (and/or prophylactically) effective amount of the composition as described herein may be administered.

As used herein, an “effective amount” is an amount sufficient to exert beneficial or desired results. Accordingly, a “therapeutically effective amount” (prophylactically effective amount) is an amount that, when administered to a subject in need thereof, is sufficient to exert some therapeutic (prophylactic) effect as described herein, such as, but not limited to, reduction of joint inflammation (joint inflammation is described elsewhere herein), increase in grip strength, reduction in bone erosion, and reduction in inflammatory infiltrates, among others, optionally compared to an untreated subject.

An amount that is “therapeutically effective” (and/or prophylactically effective) may vary from subject to subject, for example depending on the age, the disease progression or the overall general condition of the individual. An appropriate “therapeutically effective” (and/or prophylactically effective) amount in any individual case may be determined by the skilled person using routine experimentation, such as the methods described later herein, and/or the methods of the experimental part herein. It is understood that “effective amount” and “therapeutically effective amount” (and/or prophylactically effective amount) refer to the combined total amount of bacterial strains in the composition. Depending on the final application, the combined total amount can be the result of equal amounts of each of the bacterial strains or unequal amounts of each of the bacterial strains, in which each single strain optionally has a minimum abundance of 0.0001% of the combined total amount, preferably a minimum abundance of 0.001% of the combined total amount and more preferably a minimum abundance of 0.01% of the combined total amount.

Depending on the final application, said effective amount or therapeutically effective amount (and/or prophylactically effective amount) (which is understood to refer to the combined total amount of bacteria) can be at least 10\hat{ }2, preferably at least 10\hat{ }3, more preferably at least 10\hat{ }4, even more preferably at least 10\hat{ }5 bacteria. In some embodiments, said effective amount or therapeutically effective amount (and/or prophylactically effective amount) (which is understood to refer to the combined total amount of bacteria) can be between 10\hat{ }2 and 10\hat{ }14, preferably between 10\hat{ }3 and 10\hat{ }13, more preferably between 10\hat{ }4 and 10\hat{ }12, even more preferably between 10\hat{ }5 and 10\hat{ }11 bacteria. In some embodiments, said amount is a daily amount, i.e. an amount that is administered daily. In some embodiments, the number of bacteria means the number of live bacteria. In preferred embodiments, the number of live bacteria is expressed as the number of colony forming units (CFU).

Within the context of compositions for use, methods and uses provided herein, the composition may be administered to a subject in need as described herein.

A “subject” or “subject in need” as used herein may be a human (preferred) or a non-human animal. In some embodiments, the subject (in need) can be a healthy, asymptomatic or partially symptomatic subject, especially in the context of preventing joint inflammation, a symptom thereof, or an underlying disease or condition as described herein. In some embodiments, the subject (in need) may also suffer from or be at risk for developing any of the symptoms, diseases, and conditions described herein (for example the underlying diseases or conditions as described herein). In some embodiments, the subject (in need) may be a subject inflicted with any of the symptoms, diseases, and conditions described herein (for example the underlying diseases or conditions as described herein).

A composition as described herein can be administered according to any suitable regime. In some embodiments, the composition can be administered as a single one-time dose. In preferred embodiments, the composition can be administered to the subject as routinely or periodically, for example, the composition can be administered to the subject (approximately) once every 12 hours, 24 hours, or 48 hours; i.e. two times per day, daily (i.e. one time per day) or once every two days. In preferred embodiments, the composition may be administered daily (i.e. one time per day).

In some embodiments, a composition as described herein is administered for a period of at least 1 week, 8 days, 9 days, 10 days, 11 days, 12 days, 13 days, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, a year or more. In some embodiments, a composition as described herein is administered for life.

Within the context of compositions for use, methods and uses provided herein, a composition may be administered by different administration modes. In some embodiments, an administration mode may be rectally or orally. Oral administration is preferred.

A composition as provided herein may be directly or indirectly administered using suitable means known in the art. Improvements in means for providing an individual with a composition as described herein are anticipated, considering the progress that has already thus far been achieved. Such future improvements may of course be incorporated to achieve the desired effects as described herein.

Within the context of compositions for use, methods and uses provided herein, a composition as described herein may be used in combination with one or more therapeutic agents. Put differently, the compositions for use, methods and uses provided herein may further comprise the use of one or more therapeutic agents. In some embodiments, said therapeutic agent is selected from the group consisting of non-steroidal anti-inflammatory drugs (NSAIDs), glucocorticosteroids and disease-modifying antirheumatic drugs (DMARDs). Accordingly, in some embodiments, the compositions for use, methods and uses provided herein further comprise the use of a therapeutic agent selected from the group consisting of non-steroidal anti-inflammatory drugs (NSAIDs), glucocorticoids and disease-modifying antirheumatic drugs (DMARDs). Preferred therapeutic agents are DMARDs. Accordingly, in some embodiments, the compositions for use, methods and uses provided herein further comprise the use of a DMARD. Therapeutic agents belonging to the NSAIDs, steroid drugs and DMARDs are well-known in the art and have been described extensively, see e.g. Radu & Bungeau. Management of Rheumatoid Arthritis: An Overview. Cells 2021; 10(11): 2857, incorporated herein by reference in its entirety. Suitable examples and preferred options belonging to these classes of therapeutic agents have also already been described elsewhere in this disclosure.

A preferred therapeutic agent belonging to the DMARDs, more specifically belonging to the biologic DMARDs (bDMARDs), is a TNF inhibitor. Accordingly, in some embodiments, the compositions for use, methods and uses provided herein further comprise the use of a TNF inhibitor.

The compositions of two or more bacterial strains provided herein and the one or more therapeutic agents may be administered in a single composition or separately, preferably separately.

### Joint Inflammation

Clinically, joint inflammation is associated with joint stiffness, pain, weakness, and sometimes joint fatigue. Uniformly, the joint is tender and swollen, and often erythematous. Depending on the cause, it can affect one particular joint or be more widespread, affecting multiple joints throughout the body.

Joint inflammation can be diagnosed on the basis of means and methods commonly known in the art, e.g. on the basis of the clinical presentation, medical history and physical examination, radiographic examination (X-rays), blood tests, aspiration and examination of synovial joint fluid, and other studies. Examination of joint fluid of an inflamed joint generally reveals elevation of various markers of inflammation, such as, leukocytes (including neutrophils), antibodies, cytokines, cell adhesion molecules, and complement activation products. Synovial fluid total cell counts are usually above 5000 cells/mm\hat{ }3 in inflammatory arthritis.

Several clinical scoring systems relevant to joint inflammation are known to the skilled person. For example, particularly in the context of rheumatoid arthritis, the disease activity score (DAS) and the disease activity score using 28 joint counts (DAS28) have been developed (van Riel P. L. (2014). The development of the disease activity score (DAS) and the disease activity score using 28 joint counts (DAS28). Clinical and experimental rheumatology, 32(5 Suppl 85)). Another exemplary score for measuring inflammatory joint activity, particularly for rheumatoid arthritis, is the 7-joint ultrasound (US) based US7 score (US7S) (Backhaus et al. Evaluation of a novel 7-joint ultrasound score in daily rheumatologic practice: a pilot project. Arthritis Rheum. 2009; 61:1194-201).

Other relevant examples, particularly in the context of spondyloarthritis, are the ASDAS (Ankylosing Spondylitis Disease Activity Score) and BASDAI (Bath Ankylosing Spondylitis Activity Disease Activity Index) scores. BASDAI is described in Garrett, S. et al. (1994). A new approach to defining disease status in ankylosing spondylitis: the Bath Ankylosing Spondylitis Disease Activity Index. The Journal of rheumatology, 21(12), 2286-2291. ASDAS is described in Lukas C. et al. Development of an ASAS-endorsed disease activity score (ASDAS) in patients with ankylosing spondylitis. Ann Rheum Dis 2009; 68:18-24.

Within the context of compositions for use, methods and uses provided herein, a composition and/or a medicament as described herein may lead to a reduction in any of the above-mentioned scores. A reduction is preferably a reduction of at least one score level, alternatively at least two or three levels.

In some embodiments, joint inflammation may mean inflammation of one or more structures or tissues that form part of the joint. Accordingly, joint inflammation may refer to synovitis, bursitis and/or tendonitis.

In some embodiments, joint inflammation is chronic joint inflammation. Chronic joint inflammation can be understood as referring to long-lasting inflammation of the joints, for example joint inflammation occurring for a period longer than 1 month, 2 months, 3 months, 4 months, 5 months, or 6 months. In some embodiments, chronic joint inflammation may be joint inflammation associated with chronic inflammatory disorders, e.g. chronic inflammatory arthritis. Chronic inflammatory arthritis is otherwise known as autoimmune inflammatory arthritis or chronic autoimmune inflammatory arthritis. Autoimmune inflammatory arthritis is described in more detail elsewhere herein.

### Symptoms

In some embodiments, a symptom of joint inflammation as described herein comprises one or more of the classic symptoms of inflammation—i.e. dolor (pain), rubor (erythema), calor (warmth), tumor (swelling), and functio laesa (loss of function). In the context of this disclosure, functio laesa (loss of function) includes, for example, joint stiffness, decreased range of motion, and decreased strength.

In addition, joint inflammation is often associated with systemic complaints such as fatigue.

Within the context of compositions for use, methods, and uses provided herein, a composition and/or a medicament as described herein preferably exhibits at least one, at least two, at least three, or all of the following effects (preferably in the context of a joint):
reducing inflammation (such as reducing inflammatory infiltrates);
reducing pain;
reducing redness, warmth and/or swelling of the joint
reducing stiffness;
reducing bone erosion
improving range of motion;
improving strength (such as grip strength)

Means and methods for measuring these symptoms and effects are well-known in the art. Non-limiting examples of methods for measuring these symptoms and effects are provided in the experimental section.

In this context, “reducing” (respectively “improving”) and the like means at least a detectable decrease (respectively a detectable improvement) using an assay known to a person of skill in the art, such as assays as carried out in the experimental part. The reduction may be a decrease of at least 5%, at least 10%, at least 20%, at least 30%, at least 40%, at least 50%, at least 60%, at least 70%, at least 80%, at least 90% or at least 100%. In some embodiments, administering a composition as described herein can result in reduced joint inflammation (such as reduced inflammatory infiltrates), reduced joint pain, reduced redness, warmth and/or swelling of the joint, reduced joint stiffness, reduced bone erosion, improved range of motion, and improved strength (such as grip strength), optionally as compared to a subject with joint inflammation that is administered a placebo.

In some embodiments, any of the described effects are observed after at least 1 week, 8 days, 9 days, 10 days, 11 days, 12 days, 13 days, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, a year or more of treatment, said treatment optionally involving an amount and an administration regimen as described elsewhere herein.

### Underlying Diseases and Conditions

Joint inflammation and its symptoms as described herein may be found in a large number of diseases and conditions which are referred to in this disclosure as underlying diseases and conditions. It should therefore be understood that this disclosure also encompasses the use of compositions provided herein for treating or preventing any underlying diseases and conditions. In some embodiments, the compositions for use, uses and methods described herein are for treating or preventing joint inflammation associated with any of the underlying diseases and conditions described below. In some embodiments, the compositions for use, uses and methods described herein are for treating or preventing joint inflammation in a subject inflicted with any of the underlying diseases and conditions described below. Preferred features of underlying diseases and conditions in the context of this disclosure are described in this section.

An important group of underlying diseases and conditions are referred to medically as “arthritis”. There are more than 100 types of arthritis, all of them characterized at least by inflammation of one or more joints. Accordingly, in some embodiments, an underlying disease or condition as described herein is arthritis.

One possible way of broadly classifying arthritis is as inflammatory arthritis or non-inflammatory arthritis. A preferred form of arthritis in the context of this disclosure is inflammatory arthritis. Accordingly, in some embodiments, an underlying disease or condition as described herein is inflammatory arthritis.

Inflammatory arthritis is characterized by an overactive immune system. Inflammatory arthritis is usually associated with the classic symptoms of inflammation—i.e. dolor (pain), rubor (erythema), calor (warmth), tumor (swelling), and functio laesa (loss of function), although not all the features need to be always present. For an up-to-date overview of inflammatory arthritis, see e.g. “Poudel P, Goyal A, Lappin S L. Inflammatory Arthritis. 2022 Apr. 21. In: StatPearls [Internet]. Treasure Island (FL): StatPearls Publishing; 2022 January-. PMID: 29939526”.

Inflammatory arthritis can be diagnosed and monitored according to methods well-known in the art, for example as described in Ledingham et al. Diagnosis and early management of inflammatory arthritis BMJ 2017; 358:j3248.

Inflammatory arthritis can be further subdivided based on the etiology as infectious arthritis (also named septic arthritis), crystalline arthritis (also named crystal-induced arthritis), and autoimmune inflammatory arthritis. A preferred form of inflammatory arthritis in the context of this disclosure is autoimmune inflammatory arthritis. Autoimmune inflammatory arthritis may also be referred to as chronic inflammatory arthritis or chronic autoimmune inflammatory arthritis. Accordingly, in some embodiments, an underlying disease or condition as described herein is autoimmune inflammatory arthritis.

Exemplary organisms and infectious diseases causing infectious arthritis include Staphylococcus aureus, Streptococcus pneumoniae, Neisseria gonorrhea, anaerobic bacteria, mycobacteria species, brucellosis, Borrelia burgdorferi, sporotrichosis, coccidioidomycosis, and viruses (such as Parvovirus, Enterovirus, and Rubella).

Crystalline arthritis includes gout, pseudogout, and basic calcium phosphate (BCP) disease.

Primary examples of autoimmune inflammatory arthritis are rheumatoid arthritis (RA), juvenile idiopathic arthritis (JIA), spondyloarthritis (SpA), lupus, Sjögren syndrome and Lyme disease. Accordingly, in some embodiments, an underlying disease or condition as described herein is an autoimmune inflammatory arthritis selected from the group consisting of: rheumatoid arthritis (RA), juvenile idiopathic arthritis (JIA), spondyloarthritis (SpA), lupus, Sjögren syndrome and Lyme disease, preferably spondyloarthritis (SpA).

A preferred form of autoimmune inflammatory arthritis in the context of this disclosure is spondyloarthritis (SpA). Accordingly, in some embodiments, an underlying disease or condition as described herein is spondyloarthritis. Spondyloarthritis (SpA) is a term covering a family of diseases with similar clinical and genetic characteristics such as involvement of the axial skeleton, association with HLA-B27 antigen, typical involvement of peripheral joints (asymmetric oligoarthritis, enthesitis, dactylitis), as well as common extra-musculoskeletal manifestations such as acute anterior uveitis, psoriasis and inflammatory bowel disease (e.g. Crohn's disease or ulcerative colitis).

Spondyloarthritis includes, without limitation, axial spondyloarthritis (including non-radiographic axial SpA and radiographic axial SpA (also referred to as ankylosing spondylitis) and peripheral spondyloarthritis, psoriatic arthritis, enteropathic spondyloarthritis (i.e. spondyloarthritis which occurs in patients with inflammatory bowel diseases (IBDs)), and reactive arthritis.

In some embodiments, an autoimmune inflammatory arthritis is an IBD-associated arthritis. This term is used to describe types of inflammatory arthritis associated with IBD and includes some forms of spondyloarthritis, including psoriatic arthritis, ankylosing spondylitis, reactive arthritis, and enteropathic spondyloarthritis. Thus, in some embodiments, IBD-associated arthritis is selected from the group consisting of psoriatic arthritis, ankylosing spondylitis, reactive arthritis, and enteropathic spondyloarthritis.

Inflammatory bowel disease (IBD) is a term used to describe two conditions: Chrohn's disease (CD) and ulcerative colitis (UC). These conditions are typically caused by chronic inflammation in parts of the gastrointestinal (GI) tract. Crohn's disease may affect any part of the GI tract but more commonly affects the small intestine. Ulcerative colitis (UC) typically affects the large intestine and the rectum.

IBD can be diagnosed and monitored according to methods well-known in the art, for example as described in Maaser et al. ECCO-ESGAR Guideline for Diagnostic Assessment in IBD Part 1: Initial diagnosis, monitoring of known IBD, detection of complications, Journal of Crohn's and Colitis 2019; 13(2): 144-164K.

Advantageously, especially in the context of IBD-associated arthritis, compositions disclosed herein are also capable of treating intestinal inflammation. By being capable of counteracting both joint inflammation and intestinal inflammation, compositions described herein are particularly suitable for treating or preventing spondyloarthritis and IBD-associated arthritis.

Without wishing to be bound by any particular theory, it is believed that the above-described compositions and their uses and methods may be based on the following mechanisms:
a) stimulating growth and/or activity of one or a limited number of beneficial bacteria in the intestinal tract,
b) inhibiting growth and/or activity of one or a limited number of pathogenic bacteria in the intestinal tract,
c) relatively increasing the attachment of non-pathogenic bacteria to the mucosa of the gastrointestinal surface,
d) reducing uncontrolled uptake of antigens, pro-inflammatory molecules, bacteria or bacterial products by the gut,
e) providing anti-inflammatory activity at the intestinal surface,
f) increasing gut barrier functioning,
g) producing bacterial metabolites,
h) reducing the production of detrimental gut metabolites,
i) inducing wound repair,
or j) any combination of a) to i).

### General Information

Unless stated otherwise, all technical and scientific terms used herein have the same meaning as customarily and ordinarily understood by a person of ordinary skill in the art to which this invention belongs, and read in view of this disclosure.

### Sequence Identity

In the context of this disclosure, a nucleic acid molecule such as a nucleic acid molecule encoding a 16S rRNA gene is represented by a nucleic acid or nucleotide sequence which encodes a 16S rRNA.

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide.

Throughout this application, each time one refers to a specific nucleotide sequence SEQ ID NO (take SEQ ID NO: X as example) encoding a given protein or rRNA fragment, one may replace it by:
i. a nucleotide sequence comprising a nucleotide sequence that has at least 50%, 55%, 60%, 65%, 70%, 75%, 80%, 85%, 90%, 95%, 97% or 99% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 50%, 55%, 60%, 65%, 70%, 75%, 80%, 85%, 90%, 95%, 97% or 99% amino acid identity or similarity with an amino acid sequence encoded by a nucleotide sequence SEQ ID NO: X.

A preferred level of sequence identity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 99%.

In the context of 16S rRNA genes, due to their high degree of conservation among bacterial strains with comparable functional potential and similar taxonomic classification, preferred levels of sequence identity are at least 97%, 97.5%, 98%, 98.5%, 99%, 99.5%, 99.7%, 99.9% or 100%.

Throughout this application, each time one refers to a specific amino acid sequence SEQ ID NO (take SEQ ID NO: Y as example), one may replace it by: a polypeptide represented by an amino acid sequence comprising a sequence that has at least 50%, 55%, 60%, 65%, 70%, 75%, 80%, 85%, 90%, 95%, 97% or 99% sequence identity or similarity with amino acid sequence SEQ ID NO: Y. A preferred level of sequence identity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated herein by reference.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference). Alternatively percentage similarity or identity may be determined by searching against public databases, using algorithms such as FASTA, BLAST, etc. Thus, the nucleic acid and protein sequences of some embodiments of the present disclosure can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference. BLAST nucleotide searches can be performed with the NBLAST program, score=100, wordlength=12 to obtain nucleotide sequences homologous to nucleic acid molecules of the disclosure. BLAST protein searches can be performed with the BLASTx program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25(17): 3389-3402, incorporated herein by reference. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/.

Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged | Ser (S), Thr (T), Asn (N), and Gln (Q) |
| Residues | |
| Aliphatic Uncharged | Gly (G), Ala (A), Val (V), |
| Residues | Leu (L), and Ile (I) |
| Non-polar Uncharged | Cys (C), Met (M), and Pro (P) |
| Residues | |
| Aromatic Residues | Phe (F), Tyr (Y), and Trp (W) |

### Alternative Conservative Amino Acid Residue Substitution Classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, |
| | R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in | A, C, D, E, G, H, K, N, |
| turn formation | Q, R, S, P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

In this document and in its claims, the verb “to comprise” and its conjugations is used in its non-limiting sense to mean that items following the word are included or contained, but items not specifically mentioned are not excluded. Thus, the terms ‘comprising’, ‘comprises’, ‘comprised of’ and the like as used herein are synonymous with ‘including’, ‘includes’ or ‘containing’, ‘contains’, and are inclusive or open-ended and do not exclude additional, non-recited members, elements or method steps.

In addition, the verb “to consist” may be replaced by “to consist essentially of” meaning that a composition as described herein may comprise additional component(s) than the ones specifically identified, said additional component(s) not altering the unique characteristic of this disclosure. In addition, the verb “to consist” may be replaced by “to consist essentially of” meaning that a method as described herein may comprise additional step(s) than the ones specifically identified, said additional step(s) not altering the unique characteristic of this disclosure.

As used herein, the singular forms ‘a’, ‘an’, and ‘the’ include both singular and plural referents unless the context clearly dictates otherwise; for example, “a bacterial strain,” is understood to represent one or more bacterial strains. As such, the terms “a” (or “an”), “one or more,” and “at least one” can be used interchangeably herein. As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments described herein are capable of operation in other sequences than described or illustrated herein.

The word “about” or “approximately” when used in association with a numerical value (e.g. about 10) preferably means that the value may be the given value (of 10) more or less 1% of the value.

As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated. Titles, subtitles, headings and the likes are used herein solely for ease of reading and are not intended to limit or restrict the disclosure in any way.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

One skilled in the art will recognize many methods and materials similar or equivalent to those described herein, which could be used in the practice of the present invention. Indeed, the present invention is in no way limited to the methods and materials described. It is also understood that the disclosure encompasses the generalization of aspects of the following examples to the preceding disclosure.

The present invention is further described by the following examples which should not be construed as limiting the scope of the invention.

### DESCRIPTION OF THE FIGURES

FIG. 1. Body weight gain in the vehicle-treated groups. Data are presented as mean+/−SEM. Statistical differences were assessed by use of a two-way repeated measures ANOVA. VEH: vehicle.

FIG. 2. Body weight gain after gavage with vehicle or MH006. BW was measured in WT (A), heterozygous (B), and homozygous (C) mice. Data are presented as mean+/−SEM. Statistical differences were assessed by use of a two-way repeated measures ANOVA. VEH: vehicle.

FIG. 3. Arthritis development in vehicle-treated mice. A) Grip Strength Meter (BIOSEB). B) Grip strength measured throughout the course of the experiment in the vehicle-treated animals; data are shown relatively to the initial grip. C) Arthritis Clinical Score measured throughout the course of the experiment in the vehicle-treated animals. All data are presented as mean+/−SEM. Statistical differences were assessed by use of a two-way repeated measures ANOVA. VEH: vehicle.

FIG. 4. Arthritis assessment. Effect of MH006 on arthritis development. A-C) Grip strength measured throughout the course of the experiment; data are shown relatively to the initial grip. D-F) arthritis clinical score measured throughout the course of the experiment. All data are presented as mean+/−SEM. Statistical differences were assessed by use of a two-way repeated measures ANOVA. VEH: vehicle.

FIG. 5. Effect of MH006 on histological arthritis score. A-B) Different regions of the ankle were scored, as assessed at the end of the experiment. C) Details on the calcaneous erosion score. Scoring was performed by two independent observers in two slides/animal. All data are presented as mean of 2 slides, 2 readers+/−SEM. Statistical differences were assessed by use of an ordinary one-way ANOVA with Tukey's multiple comparisons (on C). Sec: Synovio-entheseal complex; TTC: Tibia, Talus, Calcaneus; VEH: Vehicle.

FIG. 6. Serum TNFalpha concentrations in vehicle treated animals. Data are presented as mean+/−SEMS. Statistical differences were assessed by use of a mixed-effects model (REML). VEH: vehicle.

FIG. 7. Serum TNFalpha concentrations upon treatment with vehicle or MH006. Systemic levels of TNFα were determined in WT (A), heterozygous (B), and homozygous (C) mice. Data are presented as mean+/−SEM. Statistical differences were assessed by use of a Mixed-effects model (REML). Note that TNFα levels are not available for all mice: N-values for WT-VEH (2-5), WT-MH006 (6-7), TNF^{ΔARE/+}—VEH (5-6), TNF^{ΔARE/+}—MH006 (3-4), TNF^{ΔARE/ARE}—VEH (5), TNF^{ΔARE/ARE} (6). VEH: vehicle.

FIG. 8. Femur length and diameter upon treatment with vehicle or MH006. The length (A) and the antero-posterior (B) and medio-lateral (C) diameters of the femur were measured. Data are presented as mean+/−SEM. Statistical differences were assessed by use of an ordinary one-way ANOVA with Tukey's multiple comparisons. VEH: Vehicle.

FIG. 9. Metabolic profile at passage 3 (P3). Net production levels of acetate, propionate, butyrate, valerate, lactate and succinate are shown. See also Table 2.

FIG. 10. Cell viability (RFU) after IL-1beta stimulation. BGM: bacterial growth medium.

FIG. 11. IL-6 secretion after IL-1beta stimulation. BGM: bacterial growth medium. One-way ANOVA, Dunnett multiple comparison testing compared to Ctrl, Compared to IL1-beta.

### EXAMPLES

### Example 1: Mouse Model

In this study we investigated the therapeutic potential of a composition comprising Phascolarctobacterium faecium, Veillonella atypica, Agathobacter rectalis, Lactiplantibacillus plantarum and Anaerostipes caccae in joint inflammation. The tested strains are Phascolarctobacterium faecium LMG P-32922, Veillonella atypica LMG P-32913, Agathobacter rectalis DSM 34506, Lactiplantibacillus plantarum LMG P-29366 and Anaerostipes caccae LMG P-29359. Said composition comprising Phascolarctobacterium faecium LMG P-32922, Veillonella atypica LMG P-32913, Agathobacter rectalis DSM 34506, Lactiplantibacillus plantarum LMG P-29366 and Anaerostipes caccae LMG P-29359 may also be denoted herein as MH006.

For this purpose, compositions of this disclosure have been tested using a new TNF-driven mouse inflammation model by targeting the AU-Rich element (ARE) of the Tnf gene using a double guide-RNA mediated CRISPR/Cas9 approach. These mice are referred to herein as TNF-DARE or TNFΔARE mice (see also Kontoyiannis et al. Immunity 1999; 10:387-398; Kontoyiannis et al. JEM 2002; 196:1563-74). TNF-DARE mice lack the AU-rich elements (ARE) in the Tnfa gene. ARE elements are involved in the destabilization of the mRNA. Therefore, deletion of the ARE regulating elements leads to increased TNFalpha levels.

TNF-DARE mice express deregulated mouse TNF that leads to the gradual development of spontaneous inflammatory polyarthritis (chronic inflammatory arthritis) and inflammatory bowel disease (ileitis). The co-incidence of arthritis and IBD in the TNF-DARE mouse closely resembles the phenotype manifested in human spondyloarthropathies. TNF-DARE mice are therefore considered as a suitable SpA model (Vieira-Sousa et al. Arthritis & Rheumatology 2015; 67(11): 2813-2827).

### Materials and Methods

The composition for administration to mice is obtained by resuspension of a lyophylized composition comprising Phascolarctobacterium faecium LMG P-32922, Veillonella atypica LMG P-32913, Agathobacter rectalis DSM 34506, Lactiplantibacillus plantarum LMG P-29366 and Anaerostipes caccae LMG P-29359. The lyophylized composition comprises a serum-based lyoprotectant.

Wild type (WT), homozygous TNF^{ΔARE/ΔARE} and heterozygous TNF^{ΔARE/+} male and female mice, about 8 weeks old, were administered one dose of streptomycin (20 mg/mouse in 100 μl) 1 day before treatment to enhance bacterial engraftment. The composition was administered by intragastric gavage (200 μL/gavage; 10\hat{ }9 CFU/dose), 1×/day and this 3×/week, except during the first week after antibiotics administration. During this first week, the composition was administered daily. Control mice (vehicle groups) were gavaged with the carrier, prepared in the same manner as the composition and administered at the same frequency as the composition. Animals were treated with either the composition or carrier for a total of 10 weeks.

Body weight; grip strength (BIOSEB) and arthritis development were macroscopically evaluated throughout the experiment. For evaluation of arthritis development, a clinical arthritis score as described in Jacques et al. was used (Jacques et al. Arthritis & Rheumatology 2010; 62(4): 988-999). Signs of arthritis were scored as follows: 0=normal, 0.5=1 finger/toe affected, 1=swelling of wrist/sagging of footpad, 2=mild swelling and distortion of the metacarpophalangeal/tarsophalangeal joints, and 3=distortion of all joints. The left femur was isolated and length/diameter was measured with a digital caliper.

After 10 weeks, mice were euthanized and blood was collected by cardiac puncture. Serum was prepared and TNFalpha levels were measured by ELISA. Ankles and ileum were collected for histological examinations (4% formaldehyde for fixation, 70% EtOH for storage).

Histological evaluation of arthritis was performed as described in Jacques et al. Arthritis & Rheumatology 2010; 62(4): 988-999: Arthritis of the hind paws was evaluated histologically for 3 parameters: inflammation (0=none, 1=limited, and 2=extensive), cartilage destruction (0=none, 1=irregular cartilage surface, 2=thinner cartilage, and 3=loss of cartilage), and bone erosion (0=none, 1=small erosions, 2=larger erosions without cortical breakthrough, and 3=large erosions involving important pieces of bone with cortical breakthrough). A composite score for arthritis was obtained from these 3 parameters.

### Results

Animal's wellbeing was monitored throughout the experiment by examining bodyweight (BW).

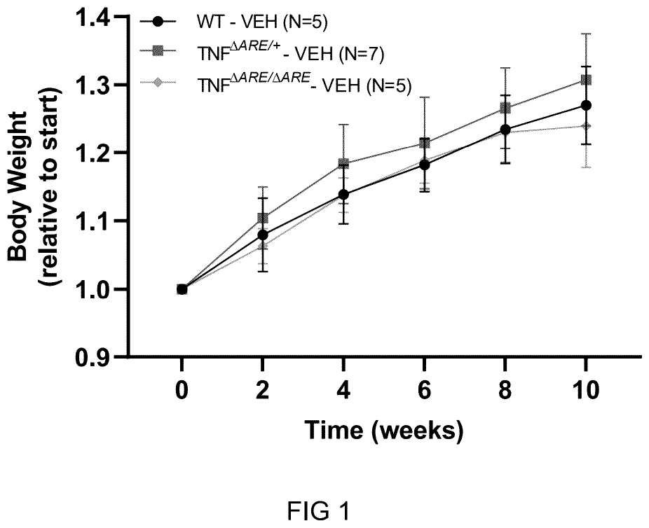

FIG. 1 depicts the body weight gain relatively to the start of treatment in the vehicle-treated groups. Despite developing ileitis and arthritis, animals did not lose weight or displayed delayed growth. No differences were detected between WT, heterozygous and homozygous animals.

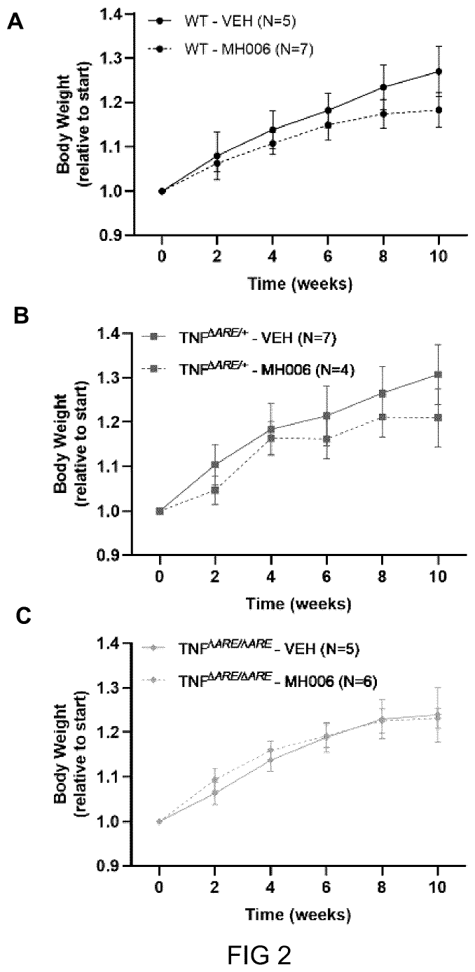

In FIG. 2, a small but insignificant effect of the tested composition was observed on BW of WT and heterozygous mice. The BW of homozygous mice was not affected. Therefore, we conclude that the tested composition, at least based on BW, does not have either a protective or detrimental effect.

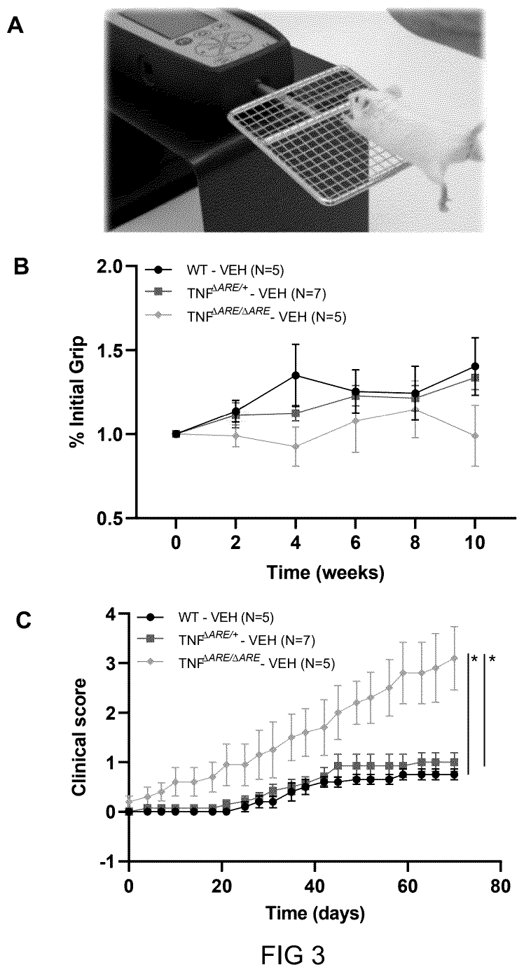

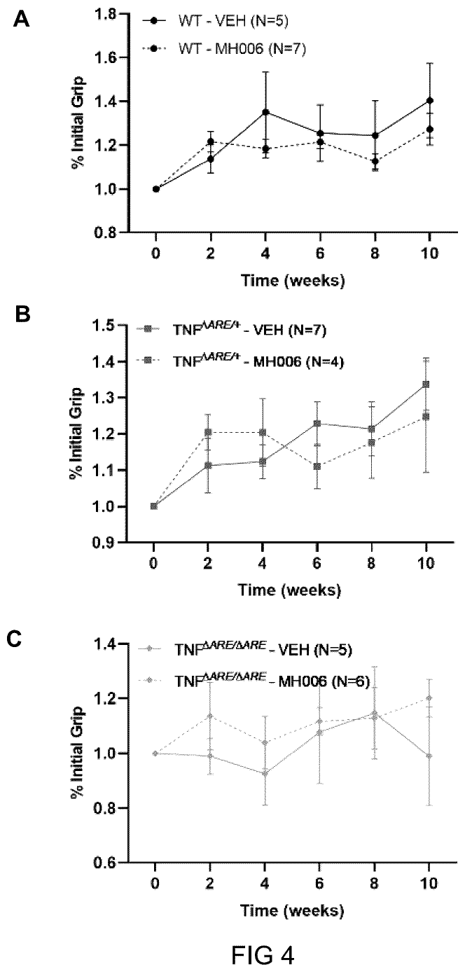

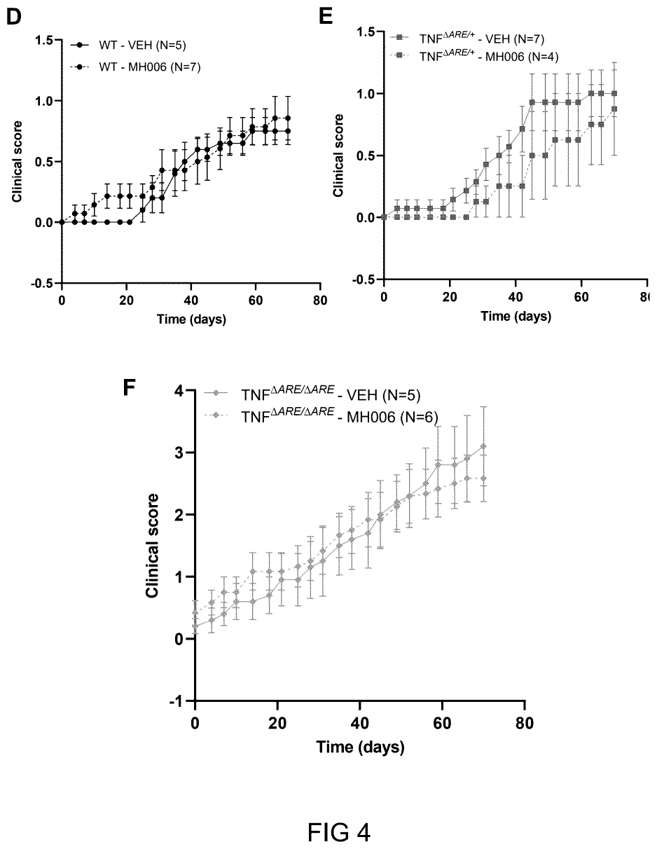

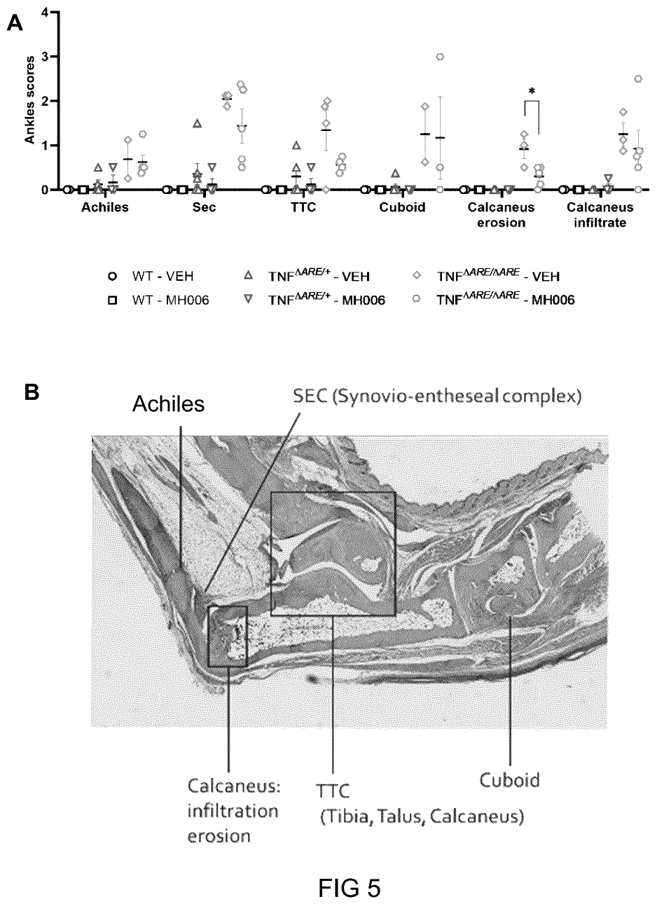

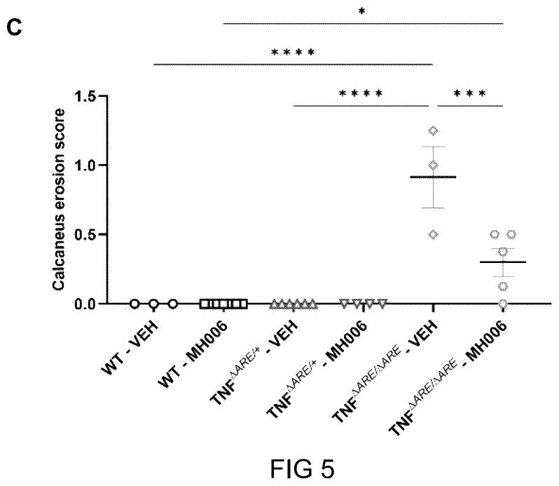

To investigate the effect of the composition on arthritis, grip strength was measured (FIG. 4 A-C) and arthritis scored in both front and hind paws (FIG. 4D-F) throughout the course of the experiment. Additionally, histological stainings of the ankles were performed to assess both erosion and inflammatory cells infiltration in different regions of the ankle (FIG. 5). In what concerns grip strength, FIG. 3 shows the results obtained for the vehicle-treated groups. Although grip strength decreases with disease severity (WT>heterozygous>homozygous), an effect that is maintained throughout the entire treatment period, no statistically significant differences were found (FIG. 3B). In contrast, and as expected, the arthritis clinical score is higher in the homozygous animals; in general, scores are significantly higher when compared to WT and heterozygous mice (FIG. 3C).

Data demonstrate a therapeutic potential for the tested composition in the treatment of joint inflammation (arthritis), with trends towards increased grip strength (FIG. 4), decreased arthritis clinical scores (FIG. 4) and decreased arthritis scores in the ankles at histopathology (FIG. 5). More specifically:
In the treated groups, grip strength tended to improve in the initial phase of the treatment, an effect that might reflect the initial daily administration of the composition. This was observed in both WT and heterozygous mice (FIG. 4A-B), whereas homozygous mice (FIG. 4C) tended to profit more during the whole course of the experiment.
In addition, positive effects upon treatment with the tested composition were observed in the arthritis clinical score; however, whereas this positive effect was observed from start to end in the heterozygous mice (FIG. 4E), in the homozygous mice the increase in clinical score only slowed down towards the end of the experiment (FIG. 4F). Important to highlight is that disease development is more severe in homozygous mice, which is clear from the clinical scores (see also FIG. 3C). This is also evident in the arthritis scores at histopathology, where almost no erosion/inflammation of the different regions of the ankle were observed in heterozygous mice, whereas homozygous mice do have a clear disease at histology (FIG. 5). Also, at histological level, the tested composition protected animals from disease development, but this effect was only observed in the homozygous mice. This was statistically significant for the erosion of the calcaneus (FIG. 5C). On the other hand, the lack of effect in the heterozygous mice can thus be explained by the lack of histological signs of arthritis in these mice.

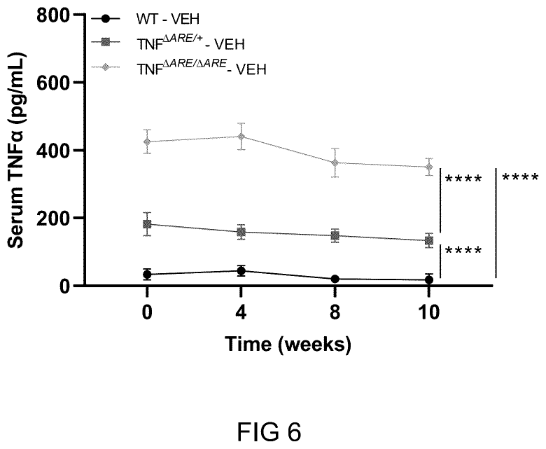

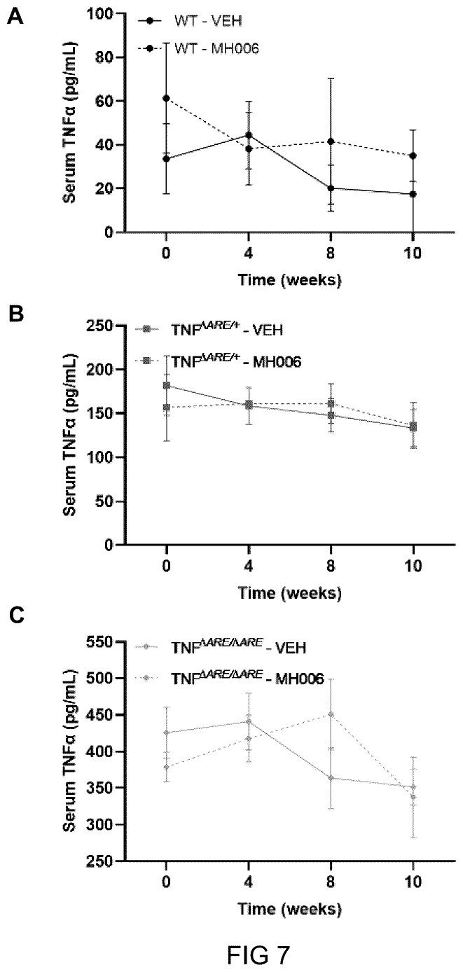

Since the TNFΔARE model is TNFalpha driven, and because TNFalpha plays an important role in SpA (as exemplified by the success of TNF-blocking therapies) we measured TNFalpha levels systemically. FIG. 6 confirms that TNFα levels increase in vehicle-treated animals with disease severity (WT<heterozygous<homozygous). However, in general, the tested composition did not change TNFα levels systemically (FIG. 7). Nevertheless, because the increase in TNFα is genetically driven, it is reasonable to expect lack of effect.

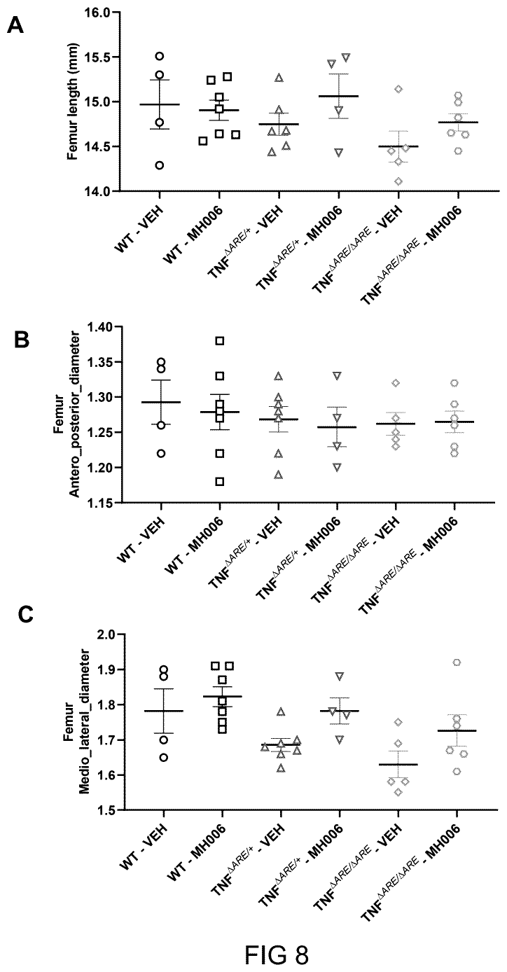

Finally, the femur was isolated, and the length and diameter measured (FIG. 8). As observed from FIG. 8, there is a decrease in all parameters (length/diameter, strength) with disease severity (WT>heterozygous>homozygous). Although changes are small, there is a clear trend for increasing the length of the femur and its medio-lateral diameter in the groups treated with MH006 (FIGS. 8A and C).

Together, the available data shows that compositions disclosed herein have preventive and therapeutic effects on the joint inflammation/arthritis phenotype in a relevant mouse model.

### Example 2: Metabolic Profiles

In Example 1, a five-strain composition of Phascolarctobacterium faecium, Veillonella atypica, Agathobacter rectalis, Lactiplantibacillus plantarum, and Anaerostipes caccae was tested. In this example, compositions comprising two, three, or four strains selected from Phascolarctobacterium faecium, Veillonella atypica, Agathobacter rectalis, Lactiplantibacillus plantarum, and Anaerostipes caccae are tested and compared to the five-strain composition and the individual strains.

A semi-continuous passaging experiment was conducted. Single strains were cultivated in growth medium under strictly anaerobic conditions. After 24 hours of growth, the single strains were combined into naive consortia using equal volumes of culture per strain. The following combinations of strains were tested:

TABLE 1
tested consortia.

| CC0101 | CC0102 | CC0103 | CC0104 |
|---|---|---|---|
| Anaerostipes | Anaerostipes | Anaerostipes | Anaerostipes |
| caccae | caccae | caccae | caccae |
| LMG P-29359 | LMG P-29359 | LMG P-29359 | LMG P-29359 |
| Veillonella | Veillonella | Veillonella | Veillonella |
| atypica | atypica | atypica | atypica |
| LMG P-32913 | LMG P-32913 | LMG P-32913 | LMG P-32913 |
| | Phascolarctobacterium | Phascolarctobacterium | Phascolarctobacterium |
| | faecium | faecium | faecium |
| | LMG P-32922 | LMG P-32922 | LMG P-32922 |
| | | Agathobacter | |
| | | rectalis | |
| | | DSM 34506 | |
| | | | Lactiplantibacillus |
| | | | plantarum |
| | | | LMG P-29366 |

Each single strain as well as each combination of strains was then inoculated at 10% (v/v) into fresh BCM medium and inoculated for 24 hours, before it was subcultured (50% v/v) to a tube containing fresh medium (=passage). This step was repeated for 3 passages in total. At each passage endpoints analysed were the production of SOFA and the total intact cell count. SOFA levels were measured by gas chromatography after liquid-liquid extraction; total cell counts were measured by flow cytometry.

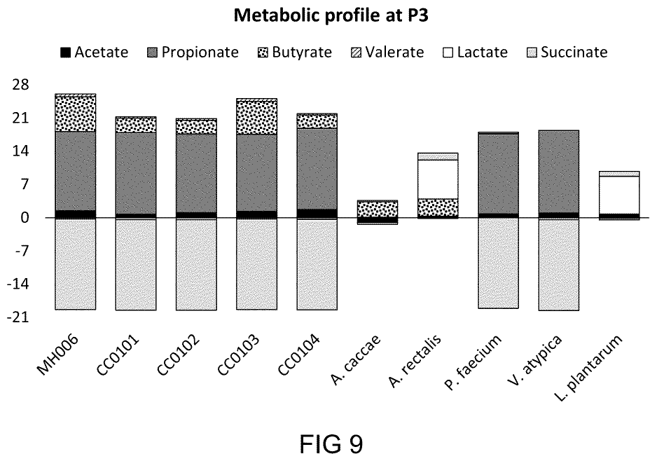

After three passages, a clear difference can be seen between the metabolic output of the strain combinations (MH006, 000101, 000102, 000103, 000104) versus the individual strains (FIG. 9 and Table 2). Cell counts at the end of each passage are shown in Table 3. All tested combinations show a globally similar SOFA profile, including significant levels of acetate, propionate, butyrate and valerate. These metabolic profiles indicate that the results described in Example 1 can be extended to compositions comprising two, three, or four strains selected from Phascolarctobacterium faecium, Veillonella atypica, Agathobacter rectalis, Lactiplantibacillus plantarum, and Anaerostipes caccae.

TABLE 2
Net metabolic output: concentrations (mM) of formed metabolites were measured
at passage 3 (P3). Media background was subtracted to highlight net production,
or consumption of metabolites (e.g. succinate consumption was measured
as succinate was added to the growth medium). See also FIG. 9.

| | Acetate | Propionate | Butyrate | Valerate | Lactate | Succinate |
|---|---|---|---|---|---|---|
| MH006 | 1.44 | 16.77 | 7.27 | 0.6 | −0.26 | −19.13 |
| CC0101 | 0.71 | 17.19 | 3.12 | 0.29 | −0.41 | −19.12 |
| CC0102 | 1.08 | 16.54 | 2.95 | 0.33 | −0.38 | −19.12 |
| CC0103 | 1.31 | 16.24 | 6.99 | 0.6 | −0.25 | −19.12 |
| CC0104 | 1.75 | 17.19 | 2.74 | 0.3 | −0.33 | −19.14 |
| A. caccae | −0.97 | 0.11 | 3.27 | 0 | −0.38 | 0.25 |
| A. rectalis | 0.31 | −0.12 | 3.63 | 0 | 8.19 | 1.45 |
| P. faecium | 0.78 | 16.9 | 0.14 | 0 | 0.25 | −19.12 |
| V. atypica | 1 | 17.45 | 0 | 0 | −0.41 | −19.13 |
| L. plantarum | 0.64 | −0.47 | 0.15 | 0 | 7.89 | 1.1 |

TABLE 3
Cell count at the end of each passage (P). Cell counts
are normalized to MH006 cell count (set at 100).

| | intact cell | intact cell | intact cell | intact cell |
|---|---|---|---|---|
| | count at 24 h | count at P1 | count at P2 | count at P3 |
| MH006 | 100,00 | 100,00 | 100,00 | 100,00 |
| CC101 | 15,34 | 14,53 | 15,14 | 20,27 |
| CC102 | 24,94 | 22,49 | 25,11 | 22,13 |
| CC103 | 87,02 | 93,49 | 100,36 | 111,31 |
| CC104 | 35,14 | 25,47 | 20,54 | 23,10 |

In view of the above, it is clear that none of the individual strains have a metabolic outbut that is similar to the metabolic output of any of the strain combinations. Accordingly, combinations of two or more bacterial strains selected from Phascolarctobacterium faecium, Veillonella atypica, Agathobacter rectalis, Lactiplantibacillus plantarum, and Anaerostipes caccae, such as 000101, CC0102, CC0103, and 000104, display unique metabolic characteristics that cannot be predicted on the basis of the characteristics of the individual strains.

### Example 3: IL-6

For the same strains and consortia as tested in Example 2, an effect on the production of IL-6 after stimulation with IL1 was investigated. 5,000 SW982 synovial fibroblasts (ATCC) were seeded in a 384-well plate. After 24 hours, cells were stimulated with 0.5 ng/mL Il1β (Invivogen, rcyec-hil1b) in combination with a 1/15 dilution of bacterial supernatant (passage 3, centrifuged and filter-sterilized). 2,500 nM ACHP Hydrochloride (IKK-2 inhibitor, MedChemExpress, HY-13060), 0,025% DMSO (ACHP solvent present on cells) and the bacterial growth medium (BCMmod4, 1/15 dilution) were used as assay controls. Cells were co-treated for 6 hours after which cell viability (Cell Titer Fluor, Promega, G6082) and IL-6 secretion (HTRF Human IL-6 Detection kit, Revvity, 62HIL06PEG) were measured.

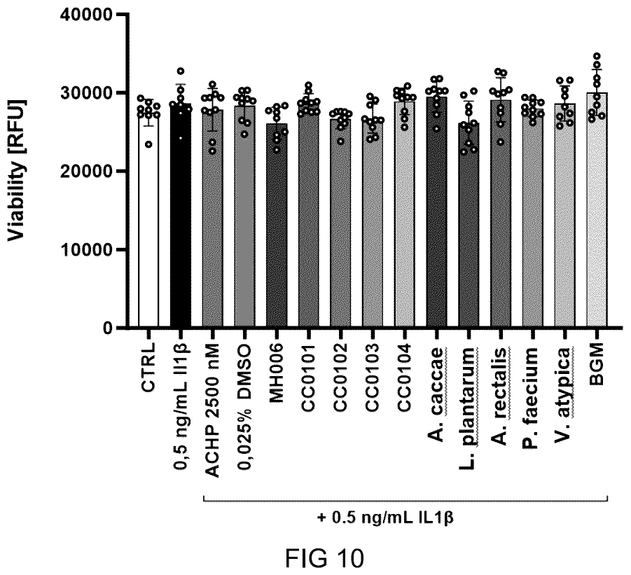

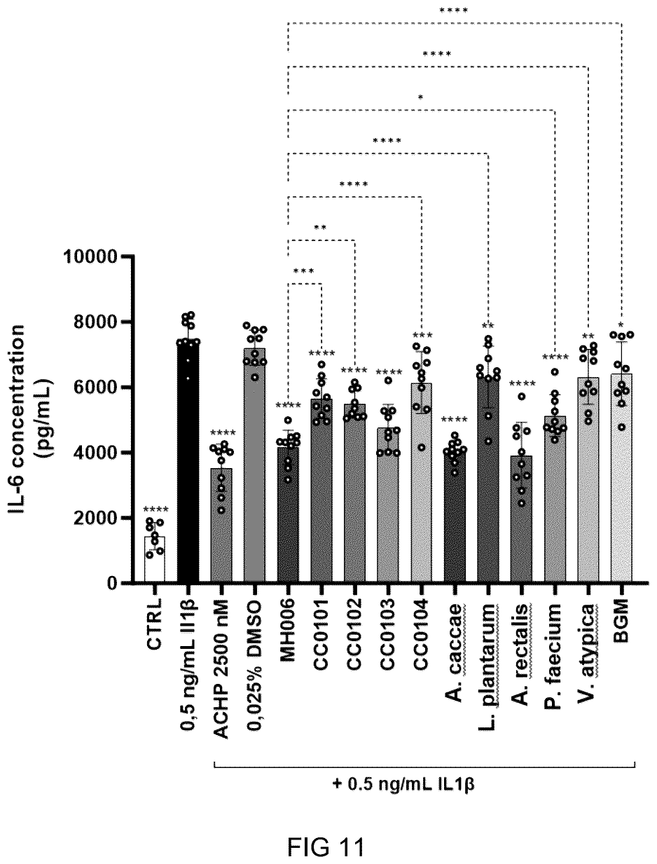

Results are summarized in FIGS. 10 (viability) and 11 (IL-6 reduction). All compositions, particularly MH006 and 000103, induced a reduction in IL-6 production.

Sequences

| SEQ ID NO: | Description of the sequence |
|---|---|
| 1 | Phascolarctobacterium faecium |
| | LMG P-32922 16S rRNA |
| 2 | Veillonella atypica |
| | LMG P-32913 16S rRNA |
| 3 | Agathobacter rectalis |
| | DSM 34506 16S rRNA |
| 4 | Lactiplantibacillus plantarum |
| | LMG P-29366 16S rRNA |
| 5 | Anaerostipes caccae |
| | LMG P-29359 16S rRNA |
| 6 | Agathobacter rectalis |
| | LMG P-32914 16S rRNA |
| 7 | Agathobacter rectalis |
| | LMG P-32915 16S rRNA |

1.-21. (canceled)

22. A method of treating or preventing joint inflammation, a symptom thereof, or an underlying disease or condition, the method comprising administering a composition of two or more bacterial strains, wherein the composition comprises a bacterial strain of at least two of the following genera: Phascolarctobacterium, Veillonella, Eubacterium, Agathobacter, Lactiplantibacillus, and Anaerostipes.

23. The method of claim 22, wherein the composition comprises a bacterial strain of at least two of the following species: Phascolarctobacterium faecium, Veillonella atypica, Agathobacter rectalis, Lactiplantibacillus plantarum, and Anaerostipes caccae.

24. The method of claim 22, wherein the composition comprises a bacterial strain of the genus Veillonella and a bacterial strain of the genus Anaerostipes, optionally wherein the composition comprises a bacterial strain of the species Veillonella atypica and a bacterial strain of the species Anaerostipes caccae.

25. The method of claim 22, wherein the composition comprises a bacterial strain of at least three or at least four of the following genera: Phascolarctobacterium, Veillonella, Eubacterium, Agathobacter, Lactiplantibacillus, and Anaerostipes.

26. The method of claim 22, wherein the composition comprises a bacterial strain of at least three or at least four of the following species: Phascolarctobacterium faecium, Veillonella atypica, Agathobacter rectalis, Lactiplantibacillus plantarum, and Anaerostipes caccae.

27. The method of claim 22, wherein the composition comprises a bacterial strain of the genus Veillonella, a bacterial strain of the genus Anaerostipes, and a bacterial strain of the genus Phascolarctobacterium, optionally wherein the composition comprises a bacterial strain of the species Veillonella atypica, a bacterial strain of the species Anaerostipes caccae, and a bacterial strain of the species Phascolarctobacterium faecium.

28. The method of claim 27, wherein the composition further comprises a bacterial strain of the genus Agathobacter, optionally wherein the composition further comprises a bacterial strain of the species Agathobacter rectalis.

29. The method of claim 27, wherein the composition further comprises a bacterial strain of the genus Lactiplantibacillus, optionally wherein the composition further comprises a bacterial strain of the species Lactiplantibacillus plantarum.

30. The method of claim 22, wherein the composition comprises a bacterial strain of at least five of the following genera: Phascolarctobacterium, Veillonella, Eubacterium, Agathobacter, Lactiplantibacillus, and Anaerostipes, optionally wherein the composition comprises a bacterial strain of each of the following genera: Phascolarctobacterium, Veillonella, Agathobacter, Lactiplantibacillus, and Anaerostipes.

31. The method of claim 22, wherein the composition comprises a bacterial strain of each of the following species: Phascolarctobacterium faecium, Veillonella atypica, Agathobacter rectalis, Lactiplantibacillus plantarum, and Anaerostipes caccae.

32. The method of claim 22, wherein the composition comprises Phascolarctobacterium faecium LMG P-32922, Veillonella atypica LMG P-32913, Agathobacter rectalis DSM 34506, Lactiplantibacillus plantarum LMG P-29366, and Anaerostipes caccae LMG P-29359.

33. The method of claim 22, wherein the underlying disease or condition is inflammatory arthritis.

34. The method of claim 33, wherein the inflammatory arthritis is autoimmune inflammatory arthritis.

35. The method of claim 34, wherein the autoimmune inflammatory arthritis is selected from the group consisting of: rheumatoid arthritis (RA), juvenile idiopathic arthritis (JIA), spondyloarthritis (SpA), lupus, Sjögren syndrome, and Lyme disease.

36. The method of claim 34, wherein the autoimmune inflammatory arthritis is spondyloarthritis (SpA).

37. The method of claim 22, further comprising administering a therapeutic agent selected from the group consisting of non-steroidal anti-inflammatory drugs (NSAIDs), glucocorticoids, and disease-modifying antirheumatic drugs (DMARDs).

38. A composition comprising a bacterial strain of each of the following genera: Phascolarctobacterium, Veillonella, Agathobacter, and Anaerostipes, optionally comprising a bacterial strain of each of the following species: Phascolarctobacterium faecium, Veillonella atypica, Agathobacter rectalis, and Anaerostipes caccae.

39. A composition according to claim 38, comprising Phascolarctobacterium faecium LMG P-32922, Veillonella atypica LMG P-32913, Agathobacter rectalis DSM 34506, and Anaerostipes caccae LMG P-29359.

40. A composition comprising a bacterial strain of each of the following genera: Phascolarctobacterium, Veillonella, Agathobacter, Lactiplantibacillus and Anaerostipes, optionally comprising a bacterial strain of each of the following species: Phascolarctobacterium faecium, Veillonella atypica, Agathobacter rectalis, Lactiplantibacillus plantarum, and Anaerostipes caccae.

41. A composition according to claim 40, comprising Phascolarctobacterium faecium LMG P-32922, Veillonella atypica LMG P-32913, Agathobacter rectalis DSM 34506, Lactiplantibacillus plantarum LMG P-29366, and Anaerostipes caccae LMG P-29359.


SEQUENCE LISTING (7 sequences)

SEQ ID NO: 1 - DNA, 1274 bp, Phascolarctobacterium faecium
  source 1..1274: mol_type = other DNA
tcttagtggcgaacgggtgagtaacgcgtaggcaacctaccctttagacggggacaacattccgaaaggagtgctaataccggatgtgatcatcgtgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagckttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagt

SEQ ID NO: 2 - DNA, 1251 bp, Veillonella atypica
  source 1..1251: mol_type = other DNA
cttagtggcgaacgggtgagtaacgcgtaatcaacctgcccttcagagggggacaacagttggaaacgactgctaataccgcatacgatccaatctcggcatcgagactggatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaatggttcttgtgcgaatagtgcgaggatttgacggtaccggaatagaaagccacggctaactacgtgccagcagccgcggtaatacgtaggtggcaagcgttgtccggaattattgggcgtaaagcgcgcgcaggcggattggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcacttcgggtgggaactcatgagagactgccgcagacaatgcggaggaaggcggggatgacgtcaaatcatcatgccccttatgacctgggctacacacgtactacaatgggagttaatagacggaagcgaaaccgcgaggtggagcaaacccgagaaacactctctcagttcggatcgtaggctgcaactcgccta

SEQ ID NO: 3 - DNA, 806 bp, Agathobacter rectalis
  source 1..806: mol_type = other DNA
tctgatctgcgattactagcgattccagcttcgtgtagtcgggttgcagactacagtccgaactgagacgttatttttgagatttgctcggcttcacagctttgcttccctttgtttacgccattgtagcacgtgtgtagcccaagtcataaggggcatgatgatttgacgtcatccccgccttcctccaggttatccctggcagtctctctagagtgcccggcctgaccgctggctactaaagataagggttgcgctcgttgcgggacttaacccaacatctcacgacacgagctgacgacaaccatgcaccacctgtcactcctgctccgaagagaaggtacggttaagtaccggtcagaaggatgtcaagacttggtaaggttcttcgcgttgcttcgaattaaaccacatgctccaccgcttgtgcgggtccccgtcaattcctttgagtttcattcttgcgaacgtactccccaggtggaatacttactgcgtttgcgacggcaccgagaagcaatgcttcccaacacctagtattcatcgtttacggcgtggactaccagggtatctaatcctgtttgctccccacgctttcgagcctcagcgtcagttatcgtccagtaagccgccttcgccactggtgttcctcctaatatctacgcatttcaccgctacactaggaattccgcttacccctccgacactctagtacgacagtttccaatgcagtaccggggttgagccccgggctttcacatcagacttgccgcaccgcctgcgctccctttacacccagtaaatcc

SEQ ID NO: 4 - DNA, 1563 bp, Lactiplantibacillus plantarum
  source 1..1563: mol_type = other DNA
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctt

SEQ ID NO: 5 - DNA, 1530 bp, Anaerostipes caccae
  source 1..1530: mol_type = other DNA
atgagagtttgatcctggctcaggatgaacgctggcggcgtgcttaacacatgcaagtcgaacgaagcatttaggattgaagttttcggatggatttcctatatgactgagtggcggacgggtgagtaacgcgtggggaacctgccctatacagggggataacagctggaaacggctgctaataccgcataagcgcacagaatcgcatgattcagtgtgaaaagccctggcagtataggatggtcccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaacagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctggtcttgacatcccaatgaccgaaccttaaccggttttttctttcgagacattggagacaggtggtgcatggttgtcgtcagctcgtgtcgtgagatgttgggttaagtcccgcaacgagcgcaacccctatctttagtagccagcattacggatgggcactctagagagactgccagggataacctggaggaaggtggggacgacgtcaaatcatcatgccccttatggccagggctacacacgtgctacaatggcgtaaacaaagggaagcgaagtcgtgaggcgaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctccttt

SEQ ID NO: 6 - DNA, 1275 bp, Agathobacter rectalis
  source 1..1275: mol_type = other DNA
tttgatttccttcgggactgattattttgtgactgagtggcggacgggtgagtaacgcgtgggtaacctgccttgtacagggggataacagttggaaacggctgctaataccgcataagcgcacagcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaatcgtaccttctcttcggagcaggagtgacaggtggtgcatggttgtcgtcagctcgtgtcgtgagatgttgggttaagtcccgcaacgagcgcaacccttatctttagtagccagcggtcaggccgggcactctagagagactgccagggataacctggaggaaggcggggatgacgtcaantcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcaga

SEQ ID NO: 7 - DNA, 1274 bp, Agathobacter rectalis
  source 1..1274: mol_type = other DNA
ttgatttccttcgggactgattattttgtgactgagtggcggacgggtgagtaacgcgtgggtaacctgccttgtacagggggataacagttggaaacggctgctaataccgcataagcgcacrgcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaycgtaccttctcttcggagcaggagtgacaggtggtgcatggttgtcgtcagctcgtgtcgtgagatgttgggttaagtcccgcaacgagcgcaacccttatctttagtagccagcggtcaggccgggcactctagagagactgccagggataacctggaggaaggcggggatgacgtcaantcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcaga